SEQUENCE LISTING (5 sequences)

SEQ ID NO: 1 - protein, 170 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 2 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 4 - protein, 629 aa, Homo sapiens [sequence omitted]

SEQ ID NO: 5 - protein, 16 aa, Homo sapiens
GGSGGSGGGGSGGGGS

(19) United States
(12) Patent Application Publication
(10) Pub. No.: US 2012/0269765 A1
(43) Pub. Date: Oct. 25, 2012

(54) CYTOKINE COMPOSITIONS AND METHODS OF USE THEREOF

(76) Inventors: K. Christopher Garcia, Menlo Park, CA (US); Patrick Lupardus, Menlo Park, CA (US); Sashank K. Reddy, Boston, MA (US); Gregory J. Sieczkiewicz, Hopkinton, MA (US)

(21) Appl. No.: 13/386,652

(22) PCT Filed: Jul. 26, 2010
(86) PCT No.: PCT/US2010/043284

§ 371 Date: Jul. 9, 2012

Related U.S. Application Data
(60) Provisional application No. 61/271,687, filed on Jul. 24, 2009.
(60) Provisional application No. 61/271,717, filed on Jul. 24, 2009.

(51) Int. Cl. A61K 38/20; C07K 19/00; C12N 15/24; C12N 15/62; C07K 16/24; A61K 39/395; A61P 29/00; A61P 17/06; A61P 19/02; A61P 1/04; A61P 25/00; C12P 21/02; C12N 5/071; C12N 5/10; C07K 14/54

(52) U.S. Cl. 424/85.2; 530/351; 536/23.5; 536/23.4; 530/389.2; 424/158.1; 435/69.52; 435/375; 435/348; 435/369; 435/328

(57) ABSTRACT

Polypeptides, including non-naturally occurring and recombinantly modified polypeptides related to the p19 subunit of IL-23, methods of making such molecules and methods of using such molecules as therapeutic, prophylactic and diagnostic agents are provided.

### CROSS REFERENCE TO RELATED APPLICATIONS

This application claims the benefit of priority under 35 U.S.C. §119(e) to U.S. Provisional Application Ser. Nos. 61/271,687 and 61/271,717, both filed on Jul. 24, 2009, and both entitled CYTOKINE COMPOSITIONS AND METHODS OF USE THEREOF, both of which are incorporated herein by reference in their entirety for all purposes.

### GOVERNMENT RIGHTS

This invention was made with government support under AI51321 awarded by National Institutes of Health. The Government has certain rights in the invention.

### BACKGROUND

The immune system protects individuals from infectious agents (for example viruses, bacteria, and multi-cellular organisms), a well as from cancer and neoplasms. The immune system includes many lymphoid and myeloid cell types such as neutrophils, monocytes, macrophages, dendritic cells (DCs), eosinophils, T cells, and B cells. These cells are capable of producing signaling proteins known as cytokines. Cytokines are soluble, small proteins that mediate a variety of biological effects, including the induction of immune cell proliferation, development, differentiation, and/or migration, as well as the regulation of the growth and differentiation of many cell types (See, for example, Arai et al., Ann. Rev. Biochem. 5P:783 (1990); Mosmann, Curr. Opin. Immunol. 5:311 (1991); Paul and Seder, Cell 76:241 (1994)). Cytokine-induced immune functions can also include an inflammatory response, characterized by a systemic or local accumulation of immune cells. Although they do have host-protective effects, these immune responses can produce pathological consequences when the response involves excessive and/or chronic inflammation, as in autoimmune disorders (such as multiple sclerosis) and cancer/neoplastic diseases (Oppenheim and Feldmann (eds.) Cytokine Reference, Academic Press, San Diego, Calif. (2001); von Andrian and Mackay New Engl. J. Med. 343:1020 (2000); Davidson and Diamond, New Engl. J. Med. 345:340 (2001); Lu et al., Mol. Cancer Res. 4:221 (2006); Dalgleish and O'Byrne, Cancer Treat. Res. 130:1 (2006)).

Proteins that constitute the cytokine group include interleukins, interferons, colony stimulating factors, tumor necrosis factors, and other regulatory molecules. For example, human interleukin-12 promotes the differentiation and function of Th1 cells that play an important role in host defenses against intracellular pathogens, but which have also been shown to contribute to autoimmune diseases. As another example, human interleukin-23 (also known as “IL-23”) is a cytokine which has been reported to promote the proliferation of T cells, in particular memory T cells and can contribute to the differentiation and/or maintenance of Th17 cells. An abundance of evidence in recent years implicates Th17 cells as central players in the pathogenesis of numerous autoimmune and inflammatory conditions.

Accordingly, the demonstrated in vivo activities of cytokines and their receptors illustrate the clinical potential of, and need for, other cytokines, cytokine receptors, cytokine agonists, and cytokine antagonists. For example, demonstrated in vivo activities of the pro-inflammatory cytokine family illustrate the enormous clinical potential of, and need for antagonists of pro-inflammatory molecules such as IL-17A and IL-23.

There is an ongoing need for new compositions useful in the prevention and treatment of diseases and disorders in mammals.

### SUMMARY OF THE INVENTION

This disclosure provides compositions and methods for the treatment of conditions associated with the cytokines IL-12 and IL-23, e.g., disorders mediated by IL-12 and/or IL-23, such as autoimmune disorders, cancer, and allergy.

IL-12 and IL-23 are key immunoregulatory cytokines that coordinate innate and adaptive immune responses. The shared use of the p40 subunit by IL-12 and IL-23 has rendered the independent targeting of these cytokines difficult. This disclosure includes a comparative analysis in identifying the structural features unique to IL-12 (p19)/p40 and IL-23 (p35)/p40 and binding surfaces and contact residues of p19/p40 and p35/p40.

In one aspect, this disclosure features p19 polypeptides in which particular amino acid residues are mutated or targeted so as to selectively interfere with the action of IL-12 or IL-23.

In some embodiments, the p19 polypeptide is engineered to be deficient in its ability to bind to the extracellular domain of IL-23R, but retains its ability to bind to the extracellular domain of IL-12Rβ1. The polypeptide can be at least 80, 85, 88, 89, 90, 91, 92, 93, 94, 95, 96, 97, 98, or 99% identity to the polypeptide of SEQ ID NO: 1 and have at least one amino acid residue mutated or deleted. For example, one or more of residues 29, 30, 31, 32, 33, 34, 35, 36, 37, 38, 39, 40, 41, 42, 43, 44, 45, 46, 47, 130, 131, 132, 133, 134, 135, 136, 137, 138, 139, 140, 141, 142, 143, 144, 145, 146, 147, 148, 149, 150, 151, and 152 are mutated or deleted. The polypeptide can be used (e.g., in a complex with a p40 subunit) to interfere with or competitively inhibit the ability of endogenous IL-12 and IL-23 from interacting with the extracellular domain of IL-12Rβ1. Accordingly, the polypeptide can be used to antagonize IL-12Rβ1 mediated signaling.

In some embodiments, the p19 polypeptide is engineered to be deficient in its ability to bind to the extracellular domain of IL-12Rβ1, but retains its ability to bind to IL-23R. The polypeptide can be at least 80, 85, 88, 89, 90, 91, 92, 93, 94, 95, 96, 97, 98, or 99% identity to the polypeptide of SEQ ID NO: 1 and have at least one amino acid residue mutated or deleted. For example, one or more of residues 10, 11, 12, 13, 14, 15, 16, 17, 18, 19, 20, 21, 22, 23, 24, 25, 26, 27, 101, 102, 103, 104, 105, 106, 107, 108, 109, 110, 111, 112, 113, 114, 115, 116, and 117 are mutated or deleted. The polypeptide can be used (e.g., in a complex with a p40 subunit) to interfere with or competitively inhibit the ability of endogenous IL-23 from interacting with the extracellular domain of IL-23R. Accordingly, the polypeptide can be used to antagonize IL-23R mediated signaling.

In some embodiments, this disclosure features a method of providing a protein. The method includes expressing an isolated nucleic acid encoding the protein(s) of the invention in a host cell and recovering the expressed protein.

In other embodiments, a composition is provided that includes a protein having an immunoglobulin heavy variable domain, and/or, an immunoglobulin light chain variable domain, wherein one and/or both of the variable domains forms an antigen binding site. The antigen binding site can bind to IL-23 p19, e.g., at an epitope that includes one or more amino acid residue(s) of SEQ ID NO: 1, for example amino acid residues 29-47 and/or, for example amino acid residues 133-140 or 141-152 of SEQ ID NO:1 or for example amino acid residues 10-27 or 101-117 of SEQ ID NO:1.

Also featured are pharmaceutical compositions that include a polypeptide described herein, e.g., a p19 polypeptide (e.g., in complex with a p40 subunit) or a protein having an antigen binding site.

In other embodiments, a method of inhibiting the production of an inflammatory mediator by a mammalian cell is provided including contacting the cell with a polypeptide that binds IL-12Rβ1 and inhibits IL-12Rβ1 and/or IL-23R signaling or a polypeptide that binds to IL-23R and inhibits IL-23R signaling.

In some embodiments, a method of preventing, and/or treating an autoimmune disorder and/or an IL-23 mediated disorder is provided. The method includes administering to a subject an effective amount of a pharmaceutical composition described herein. The autoimmune disorder can be, for example, psoriasis, psoriatic arthritis, psoriatic dermatitis, Crohn's disease, ulcerative colitis, rheumatoid arthritis, uveitis, ankylosing spondylitis, and multiple sclerosis.

Further features and advantages will now be more particularly described in the following detailed description and claims.

### BRIEF DESCRIPTION OF THE FIGURES

FIG. 1 is a schematic representation of the IL-23 and IL-12 signaling complexes. IgD stands for “Ig-like domain”, while CHR denotes “cytokine-binding homology region” and FnIII denotes “Fibronectin-type III domain”. Lines found within ovals denote two conserved disulfide linkages and the conserved WSXWS motif.

FIG. 2 is a structural model of IL-23. (a) Side view of the IL-23 crystal structure. p19 helices A to D are encircled while the p40 domains (E1-3) are not. The single N-acetylglucosamine residue attached to Asn200 on p40 is located within the dashed circle. Disulfide linkages are colored in yellow. (b) Face-on view of IL-23. Receptor interaction sites 2 and 3 are highlighted in ovals, and the key site 3-interacting side chain of Trp 137 is displayed.

FIG. 3 depicts the structural anatomy of the p19-p40 interface. (a) Close-up view of the secondary structure and amino acid contacts between p19 and p40. (b) Cut away view of the ‘Arginine pocket’ 2Fo-Fc electron density contoured at 1.5 c. Several well-ordered waters are visible at the interface, stabilizing the interaction of p40 pocket residues with Arg159.

FIG. 4 is a comparative analysis of IL-23 and IL-12. (a) p19 (A1,B1,C1,D1) is ‘rolled’ toward the E2 of p40 (E1-3) by ˜20° and (b) tilted by ˜10° when compared to p35. (c) Overlay of the p19-p40 and p35-p40 interaction interfaces when p40 is structurally superimposed. Note the intact side chain positioning of p19 Asp159 and p40 ‘Arginine pocket’ residues. All contact residues on p19 and p35 are drawn as sticks. (d) Comparison of the four-helix bundle interacting loops of p40 form the two structures reveals only slight distortions in the positioning of the loops when bound to their respective four-helix cytokines.

FIG. 5 is a comparison of shared and distinct contact surfaces in the IL-23 and IL-12 complexes. (a) Histogram of buried surface area contributed by each p40 residue involved in p19 and/or p35 interaction. (b) Surface representation of p40 (middle panel) with residues interacting with p19, p35, or p19 and p35 highlighted. The p40-interacting surfaces of p19 and p35 are shown with a transparent p40 overlay to demonstrate orientation.

FIG. 6 depicts a commassie gel of fractions taken from a gel filtration run in which IL-23 and the extracellular domain of murine IL-12R01 were coinjected. The IL-23 subunits (p19 and p23) co-migrate with the receptor subunit indicating formation of a stable ternary complex.

FIG. 7 depicts a commassie gel of fractions taken from a gel filtration run in which IL-23 and the extracellular domains of IL-23R and IL-12Rβ1 were coinjected. The IL-23 subunits (p19 and p23) co-migrate with the receptor subunits indicating formation of a stable quarternary complex.

### DETAILED DESCRIPTION

The ability to antagonize the activity of IL-12 and IL-23 offers significant advantages in the treatment of a number of disease states including autoimmune disorders, cancer and allergy. Disclosed herein are p19 polypeptides that can bind to a cell signaling receptor that responds to interleukins. Consistent with their molecular structures, the polypeptides can, for example, compete with IL-23 and/or IL-12 for receptor binding to reduce signaling by these interleukins.

In order for the present disclosure to be more readily understood, certain terms and phrases are defined below as well as throughout the specification.

Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, N.Y. 2001); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many terms used in the present disclosure. As appropriate, procedures involving the use of commercially available kits and reagents are generally carried out in accordance with manufacturer defined protocols and/or parameters unless otherwise noted.

The term “effective amount” as used herein refers to an amount necessary to elicit a desired biological response. The effective amount of a drug may vary depending on such factors as the desired biological endpoint, the drug to be delivered, the composition of any additional active or inactive ingredients, etc. In some embodiments, an effective amount refers to, for example, an amount sufficient to result in the amelioration of one or more symptoms of a disorder. In other embodiments, an effective amount refers to an amount, for example, sufficient to cause regression of a disorder or an amount to prevent further deterioration. In other embodiments, an effective amount refers, for example, to an amount sufficient to enhance or improve the therapeutic effect(s) of another therapy.

The term “weaker” as used herein refers to a reduced level of a measurable interaction between a candidate polypeptide(s) and one, two and/or more polypeptides in a given assay. The interaction is weaker if there is witnessed a reduction in the affinity of interaction by any amount such as, for example, by 2%, 5%, 10%, 15%, 25%, 35%, 45%, 55%, 65%, 75%, 85%, 95% or more, or, 1.5-fold, 2-fold, 5-fold, 10-fold, 20-fold, 25-fold, 30-fold, 35-fold, 40-fold, 45-fold, or 50-fold or more in a given assay.

The term “expression” is used herein to mean the process by which a polypeptide is produced from nucleic acid, typically DNA. The process involves the transcription of the gene into mRNA and the translation of this mRNA into a polypeptide. Depending on the context in which it is used, “expression” may refer to the production of RNA, protein, or both.

A “fragment” of a polynucleotide encoding an IL-23 polypeptide will encode at least 5, 10, 15, 25, 30, 50, 60, 70, 100, 150, or more contiguous amino acids, or up to the total number of amino acids present in a full-length subunit of an IL-23 protein, e.g. for p19 about 160-180, 168-172, 160-170, 168-170 amino acids. The length of the fragment can be appropriately changed depending on the purpose. For example, the lower limit of the length of the fragment includes 5, 6, 7, 8, 9, 10, 15, 20, 25, 30, 40, 50, 75, 100 or more amino acids. Lengths represented by integers that are not herein specified, for example, 11 and the like can be appropriate as a lower limit. Mutations, truncations, substitutions and other alterations of the sequence are included in the definition of fragment, provided some degree of biochemical activity of interest is preserved. Fragments of an IL-23-encoding polynucleotide that are useful as hybridization probes, PCR primers, or a suppression constructs generally need not encode a biologically active portion of an IL-23 protein. A biologically active portion of a polypeptide comprising an IL-23 protein, can be prepared by isolating a portion of an IL-23 polynucleotide, expressing the encoded portion of the IL-23 protein (for example, by recombinant expression in vitro), and assessing the activity of the encoded portion of the IL-23 protein. A polynucleotide that is a fragment of an IL-23 encoding nucleotide sequence contains at least 15, 45, 75, 100, 150, 200, 250, 300, 350, 400, 450, 500, 550, or 600 contiguous nucleotides, or up to the number of nucleotides present in a full-length IL-23 polynucleotide.

The term “gene product” as used herein means an RNA (for example, a messenger RNA (mRNA) or a micro RNA (miRNA)) or protein that is encoded by the gene.

As used herein, the term “isolated” and its grammatical variants refers to a material that is removed from its original environment (e.g., the cells or materials from which the material is produced). An isolated protein can be substantially pure, e.g., 60%, 65%, 70%, 75%, 80%, 85%, 90%, 95% or 99% free of other, different protein molecules.

As used herein, the terms “modulate” and “modulation” and grammatical variants refer to the down regulation (that is, inhibition or suppression), of specifically targeted genes (including their RA and/or protein products), signaling pathways, cells, and/or a targeted phenotype, or the up regulation (that is, induction or increase) of the targeted genes. Down regulation is witnessed if there is reduction by any given amount such as, for example, by 2%, 5%, 10%, 15%, 25%, 35%, 45%, 55%, 65%, 75%, 85%, 95%, or less activity, for example, no activity, in the presence of a candidate molecule relative to its absence. Up regulation is witnessed if there is a gain by any amount such as, for example, by 2%, 5%, 10%, 15%, 25%, 35%, 45%, 55%, 65%, 75%, 85%, 95%, 100% or more, or, up to 1.5-fold, 2-fold, 5-fold, 10-fold, 20-fold, 50-fold, 100-fold, or more in the presence of a candidate molecule as compared to the absence of the candidate molecule.

“Patient” or “subject” means a mammal, for example a human, who has or is at risk for developing a disease or condition such as an inflammatory disease, or has or is diagnosed as having an inflammatory disease, or could otherwise benefit from the compositions and methods described herein.

The term “reduce” and its grammatical variants as used herein refer to any inhibition, reduction, decrease, suppression, down regulation, or prevention in expression or gene product activity. For example, the level of expression or activity can be, for example, 100% or less than 100%, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression or activity.

The terms “treating” or “treatment” or “alleviation” or “amelioration” refer to both therapeutic treatment and prophylactic or preventative measures, wherein the object is to prevent or slow down (lessen) the targeted pathologic condition or disorder.

“Variants” is intended to mean substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” or “wild-type” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the IL-23 polypeptides. Naturally occurring allelic variants such as these can be identified with the use of well-known molecular biology techniques, as, for example, with polymerase chain reaction (PCR) and hybridization techniques as outlined elsewhere herein. Variant polynucleotides also include synthetically derived polynucleotides, such as those generated, for example, by using site-directed mutagenesis but which still encode a polypeptide comprising an IL-23 specific domain, or an IL-23-like polypeptide that is capable of modulating a signaling pathway. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide (e.g., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 1 is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more sites in the native protein and/or substitution of one or more amino acids at one or more sites in the native protein. Variant proteins encompassed by the present disclosure are biologically active, that is they continue to possess a desired biological activity, which may be the same as, distinct from and/or antagonistic to that of the native protein. Such variants may result from, for example, genetic polymorphism or human manipulation. Biologically active variants of an IL-23 polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the IL-23 polypeptide as determined by sequence alignment programs and parameters described elsewhere herein. A biologically active variant of may differ from that protein by as few as 1-15 amino acid residues, as few as 1-10, such as 6-10, as few as 5, as few as 4, 3, 2 or even by one amino acid residue.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 10, a gap extend penalty of 2, and a frameshift gap penalty of 4. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

The term “antibody” or “antibodies” as used herein includes any protein that includes at least one immunoglobulin variable domain, and includes for example polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, FAT fragments, single chain antibodies and the like, as well as synthetic antigen-binding peptides and polypeptides, are also included. Non-human antibodies can be humanized by grafting non-human CDRs onto human framework and constant regions, or by incorporating the entire non-human variable domains. In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Monoclonal antibodies can also be produced in mice that have been genetically altered to produce antibodies that have a human immunoglobulin gene structure.

Immunomodulatory Polypeptides

The interactions of four-helix bundle cytokines with their class I cytokine receptors control many aspects of cellular development, differentiation, and proliferation across the immune, nervous, and hematopoietic systems. Engagement of cytokines by their receptors results in receptor homo- or heterodimerization, leading to the activation of intracellular Jak/Stat signaling cascades. Leonard, W. J. & O'Shea, J. J. (1998). Ann. Rev Immunol 16:293. Four-helix bundle cytokines have a stereotypical up-up-down-down helical topology for both short and long chain members of the family. Sprang, S. R & Bazan, 1. F. (1993). Curr. Opin. Struct. Biol. 3:815. Cytokine receptors also have several conserved features, such as the presence of a ‘cytokine binding homology region’ (CHR) that is characterized by tandem Fibronectin-type III domains (FnIII) containing a hallmark pattern of disulfide bonds, and a WSXWS motif in the second of the FnIII domains. Bazan, J. F. (1990). Proc Natl Acad Sci USA 87:6934. The prototypical long chain cytokine, human Growth Hormone (hGH) was shown to homodimerize its receptors using a ‘site I/site II’ paradigm through the sides of its helices, and that the receptors engaged the cytokine through inter-strand loops at the junction of the two FnIII domains (de Vos, A. M., Ultsch, M. & Kossiakoff, A. A. (1992). Science 255:306; Wells, J. A. & de Vos, A. M. (1996). Annu Rev Biochem 65:609). While this basic building block has been found in structures of all cytokine receptor complexes, the gp130, or IL-6/IL-12 family of cytokines has evolved an additional receptor binding epitope, termed site III, that requires the presence of a top-mounted Ig-domain on the receptors to homo or heterodimerize signaling complexes in the ‘Tall’ receptor family (for example, gp130, LIF-R, IL-12, etc.) (Chow, D., et al. (2001). Science 291:2150; Boulanger, M. J. & Garcia, K. C. (2004). Adv Protein Chem 68: 107)). Recently, several orphan members of the IL-12 class of cytokines, IL-23 and IL-27, have been paired with their cognate receptors, and their biological activities clarified (Parham, C., et al. (2002). J Immunol 168:5699; Pflanz, S. et al. (2004). J Immunol 172:2225.). In particular, IL-23 has generated a great deal of excitement with regards to its potential role in immune modulation of different subpopulations of T helper cells in concert with IL-12 (Trinchieri, G., et al. (2003). Immunity. 19:641; Kastelein, R. A., et al. (2007). Ann. Review of Immunol. 25:221).

IL-12 and IL-23 are heterodimeric cytokines that, unlike typical four-helix cytokines that are secreted alone, are secreted from dendritic cells and macrophages as disulfide-linked complexes between the helical cytokines p35 and p19, respectively, and a shared binding protein termed p40 (Oppmann, B. et al. (2000). Immunity 13:715; Gubler, D., et al. (1991). PNAS 88:4143; Wolf, S. F. et al. (1991). Journal of Immunology 146:3074; Kastelein, R. et al. (2007). Annual Review of Immunology 25:221.). Both p35 and p19 have sequence homology to IL-6 and G-CSF, marking them as members of the gp130-class of long-chain cytokines, and the p40 subunit is similar in structure to typical class I cytokine receptors such as the non-signaling alpha receptors for IL-6 and CNTF (Yoon, C., et al. (2000). Embo J 19:3530.). In essence, IL-12 and IL-23 represent cytokines constitutively associated with a soluble a-receptor subunit. While many cytokines exist as naturally ‘shed’ soluble complexes with their a-receptors (Briso, E. M., et al. (2008). J. Immunol. 180:7102.), IL-12 and IL-23 are unique in that they are secreted as binary complexes.

With regard to receptor activation, while IL-12 and IL-23 share p40, they signal through different heterodimeric cell surface complexes involving receptors homologous to gp130. IL-12 (p35/p40) signals through a heterodimer consisting of IL-12Rβ1 and IL-12Rβ2 (Presky, D. H., et al. (1996). Proc Natl Acad Sci USA 93:14002.), whereas IL-23 (p19/p40) signals through a heterodimer consisting of IL-12Rβ1 and the IL-12Rβ2-like receptor called IL-23R (Parham, C., et al. (2002) J Immunol 168:5699. The term “IL-12 receptor subunit beta 1” includes polypeptides known variously as IL-12Rβ1 and IL-12Rb1, and these terms are understood to be equivalent. IL-23 activates the same spectrum of Janus kinase (Jak)/signal transducers and activators of transcription (Stat) signaling molecules as IL-12: Jak2, Tyk2, and Stat1, Stat3, Stat4, and Stat5 (Parham, 2002).

Biologically, it was previously thought likely that these two cytokines would have redundant roles in immune homeostasis, namely T helper (Th)1-type responses that are important for cell-mediated antimicrobial and cytotoxic activities, based on their shared use of p40 as a subunit. However, it was quickly shown that their functions are non-redundant (Kastelein, R. A., et al. (2007). Annual Review of Immunology 25:221.). Whereas IL-12 drives the typical Th1 responses such as interferon-γ (IFN-γ) production, IL-23 does not influence the development of Th1 cells, but instead drives the development of an alternate CD4⁺ T cell population, now termed Th17 cells, that are notable for their production of pro-inflammatory IL-17 cytokines (Aggarwal, S., et al. (2003). Journal of Biological Chemistry 278:1910.). Several studies have shown that IL-23 regulation of autoreactive Th17 cells plays a critical role in the development of chronic autoimmune disorders (Yen, D., et al. (2006). Journal of Clinical Investigation 116:1310; Murphy, C. A., et al. (2003). Journal of Experimental Medicine 198:1951; Langrish, C. L., et al. (2005). Journal of Experimental Medicine 201:233.). Recently, IL-23 has also been shown to possess tumor-promoting proinflammatory activity, and that IL-23 blockade can render tumors susceptible to infiltration by IL-12-induced cytotoxic T cells (Langowski, J. L. et al. (2006). Nature 442:461.). These studies have led to considerable interest in the possibility of therapeutic IL-23 blockade for treatment of autoimmune disorders, cancer, and other indications.

Because IL-12 and IL-23 share their p40 subunit, it is important to delineate common versus cytokine-specific protein-protein interactions to serve as guideposts for the potential development of IL-12 versus IL-23-specific antagonists. The disclosed structural analysis provides insight into the design antagonists of IL-12 and IL-23.

The present disclosure provides compositions and methods for the reduction and the inhibition of the activities of the pro inflammatory cytokines, IL-12 and IL-23. The present disclosure is based, in part, on the binding determinants that specify IL-23 binding to the receptor IL-23R and on the binding determinants that specify IL-23 binding to the shared receptor IL-12Rβ1. Since the cytokines IL-12 and IL-23 both utilize the receptor IL-12Rβ1 as part of their respective signaling complexes, antagonism of IL-12Rβ1 will reduce both IL-12 and IL-23 signaling on cells competent to respond to both cytokines. On the other hand, since only IL-23 utilizes the IL-23R, selective antagonism of IL-23R will reduce IL-23 signaling on cells competent to respond to both cytokines. Elimination of the IL-23R binding-ability of agents that are ordinarily capable of binding IL-23R and IL-12Rβ1, but are ordinarily incapable of binding IL-12Rβ2 will generate agents that antagonize IL-12Rβ1 signaling by preventing the formation of its heterodimeric signaling complexes. Cells that are competent to respond to both cytokines IL-12 and IL-23 include those cells that in addition to expressing IL-12Rβ1, also express both IL-12Rβ2 and IL-23R. On cells that in addition to expressing IL-12Rβ1, also express IL-12Rβ2 but do not express IL-23R, the antagonists described herein will block IL-12 signaling. On cells that in addition to expressing IL-12Rβ1, also express IL-23R but do not express IL-12Rβ2, the antagonists described herein block IL-23 signaling. Therefore, the present disclosure concerns the inhibition or neutralization of IL-12 or IL-23 signaling, either singly or in combination through antagonism of their shared receptor IL-12Rβ1.

Additionally, the present disclosure discloses agents that inhibit or neutralize IL-23 signaling through antagonism of the IL-23R. Elimination of the IL-12Rβ1 binding-ability of agents that are ordinarily capable of binding IL-23R and IL-12Rβ1, but are ordinarily incapable of binding IL-12Rβ2 will generate agents that antagonize IL-23R signaling by preventing the formation of its heterodimeric signaling complex. Cells that are competent to respond to both cytokines include those cells that, in addition to expressing IL-12Rβ1, also express both IL-12Rβ2 and IL-23R. On cells that, in addition to expressing IL-12Rβ1, also express IL-12Rβ2 but do not express IL-23R, the antagonists described herein do not block IL-12 signaling. On cells that in addition to expressing IL-12Rβ1, also express IL-23R but do not express IL-12Rβ2, the antagonists described herein block IL-23 signaling.

The antagonistic molecule or neutralizing entity inhibits the activity of IL-12 and IL-23 and thus, inhibits the production, maintenance, and activity of new and existing IL-17-producing T cells (Th17). Cytokines produced by Th17 cells include IL-17A and IL-17F, and can also include IL-22, IL-23, IL-26, CCL20, CCR6, RORC, RORC2, RORγt, IL-1, IL-6, IL-23R, IL-21. Furthermore, via its inhibitory effects on IL-12 signaling, the antagonistic molecule or neutralizing entity inhibits the production, maintenance, and activity of new and existing IFN-γ-producing T helper 1 cells (Th1). The application concerns the use of IL-12Rβ1 antagonists or neutralizing entities in the treatment of inflammatory diseases characterized by the presence of elevated levels of IL-17 and/or IL-23 and/or IFN-γ. The application also concerns the use of IL-12Rβ1 antagonists in the treatment of cancers and other pathologic conditions.

The application further concerns the use of IL-23R antagonists or neutralizing entities in the treatment of inflammatory diseases characterized by the presence of elevated levels of IL-17 and/or IL-23 and/or IFN-γ. The application also concerns the use of IL-23R antagonists in the treatment of cancers and other pathologic conditions.

IL-23 Polypeptides

IL-12 and IL-23 appear to represent a unique signaling system within the cytokine network that provides innovative approaches to the manipulation of immune and inflammatory responses. As such, antagonists to IL-12 and IL-23 activity either singly or together, such as the antagonists of the present disclosure (that is designer cytokine antagonists of IL-12Rβ1), are useful in therapeutic treatment of inflammatory diseases such as multiple sclerosis, inflammatory bowel disease (IBD), rheumatoid arthritis, psoriasis, and cancer. Moreover, antagonists to IL-12 and IL-23 activity, such as the antagonists of the present disclosure (that is designer cytokine antagonists of IL-12Rβ1), are useful in therapeutic treatment of other inflammatory diseases. These antagonists are capable of binding, blocking, inhibiting, reducing, antagonizing and/or neutralizing IL-12 and IL-23 (either individually or together) in the treatment of atopic and contact dermatitis, colitis, endotoxemia, arthritis, rheumatoid arthritis, psoriatic arthritis, autoimmune ocular diseases (uveitis, scleritis), adult respiratory disease (ARD), demyelinating diseases, septic shock, multiple organ failure, inflammatory lung injury such as asthma, chronic obstructive pulmonary disease (COPD), airway hyper-responsiveness, chronic bronchitis, allergic asthma, psoriasis, eczema, IBS and inflammatory bowel disease (IBD) such as ulcerative colitis and Crohn's disease, diabetes, Helicobacter pylori infection, intraabdominal adhesions and/or abscesses as results of peritoneal inflammation (that is from infection, injury, etc.), systemic lupus erythematosus (SLE), multiple sclerosis, systemic sclerosis, nephrotic syndrome, organ allograft rejection, graft vs. host disease (GVHD), kidney, lung, heart, etc., transplant rejection, streptococcal cell wall (SCW)-induced arthritis, osteoarthritis, gingivitis/periodontitis, herpetic stromal keratitis, restenosis, Kawasaki disease, atherosclerosis, diabetes mellitus, and cancers/neoplastic diseases that are characterized by IL-17 and/or IL-23 expression, including but not limited to prostate, renal, colon, ovarian and cervical cancer, and leukemias (Tartour et al, Cancer Res. 5P:3698 (1999); Kato et al., Biochem. Biophys. Res. Commun. 282:735 (2001); Steiner et al., Prostate. 56:171 (2003); Langowksi et al, Nature 442: 461, 2006).

TABLE 1
IL-23 p19 (Accession No.: NP_057668)
RAVPGGSSPAWTQCQQLSQKLCTLAWSAHPLVGHMDLREEGDEETTNDVPHIQCGDGCDPQG
LRDNSQFCLQRIHQGLIFYEKLLGSDIFTGEPSLLPDSPVGQLHASLLGLSQLLQPEGHHWE
TQQIPSLSPSQPWQRLLLRFKILRSLQAFVAVAARVFAHGAATLSP
(SEQ ID NO: 1)
IL-23 p40 (Accession No.: P29460)
MCHQQLVISWFSLVFLASPLVAIWELKKDVYVVELDWYPDAPGEMVVLTCDTPEEDGITWTL
DQSSEVLGSGKTLTIQVKEFGDAGQYTCHKGGEVLSHSLLLLHKKEDGIWSTDILKDQKEPK
NKTFLRCEAKNYSGRFTCWWLTTISTDLTFSVKSSRGSSDPQGVTCGAATLSAERVRGDNKE
YEYSVECQEDSACPAAEESLPIEVMVDAVHKLKYENYTSSFFIRDIIKPDPPKNLQLKPLKN
SRQVEVSWEYPDTWSTPHSYFSLTFCVQVQGKSKREKKDRVFTDKTSATVICRKNASISVRA
QDRYYSSSWSEWASVPCS
(SEQ ID NO: 2)
IL-12 Receptor β1 (Accession No.: P42701)
MEPLVTWVVPLLFLFLLSRQGAACRTSECCFQDPPYPDADSGSASGPRDLRCYRISSDRYEC
SWQYEGPTAGVSHFLRCCLSSGROCYFAAGSATRLQFSDQAGVSVLYTVTLWVESWARNQTE
KSPEVTLQLYNSVKYEPPLGDIKVSKLAGQLRMEWETPDNQVGAEVQFRHRTPSSPWKLGDC
GPQDDDTESCLCPLEMNVAQEFQLRRRQLGSQGSSWSKWSSPVCVPPENPPQPQVRFSVEQL
GQDGRRRLTLKEQPTQLELPEGCQGLAPGTEVTYRLQLHMLSCPCKAKATRTLHLGKMPYLS
GAAYNVAVISSNQFGPGLNQTWHIPADTHTEPVALNISVGTNGTTMYWPARAQSMTYCIEWQ
PVGQDGGLATCSLTAPQDPDPAGMATYSWSRESGAMGQEKCYYITIFASAHPEKLTLWSTVL
STYHFGGNASAAGTPHHVSVKNHSLDSVSVDWAPSLLSTCPGVLKEYVVRCRDEDSKQVSEH
PVQPTETQVTLSGLRAGVAYTVQVRADTAWLRGVWSQPQRFSIEVQVSDWLIFFASLGSFLS
ILLVGVLGYLGLNRAARHLCPPLPTPCASSAIEFPGGKETWQWINPVDFQEEASLQEALVVE
MSWDKGERTEPLEKTELPEGAPELALDTELSLEDGDRCKAKM
(SEQ ID NO: 3)
IL-23 Receptor A (Accession No.: Q5VWK5)
MNQVTIQWDAVIALYILFSWCHGGITNINCSGHIWVEPATIFKMGMNISIYCQAAIKNCQPR
KLHFYKNGIKERFQITRINKTTARLWYKNFLEPHASMYCTAECPKHFQETLICGKDISSGYP
PDIPDEVTCVIYEYSGNMTCTWNAGKLTYIDTKYVVHVKSLETEEEQQYLTSSYINISTDSL
QGGKKYLVWVQAANALGMEESKQLQIHLDDIVIPSAAVISRAETINATVPKTIIYWDSQTTI
EKVSCEMRYKATTNQTWNVKEFDTNFTYVQQSEFYLEPNIKYVFQVRCQETGKRYWQPWSSP
FFHKTPETVPQVTSKAFQHDTWNSGLTVASISTGHLTSDNRGDIGLLLGMIVFAVMLSILSL
IGIFNRSFRTGIKRRILLLIPKWLYEDIPNMKNSNVVKMLQENSELMNNNSSEQVLYVDPMI
TEIKEIFIPEHKPTDYKKENTGPLETRDYPQNSLFDNTTVVYIPDLNTGYKPQISNFLPEGS
HLSNNNEITSLTLKPPVDSLDSGNNPRLQKHPNFAFSVSSVNSLSNTIFLGELSLILNQGEC
SSPDIQNSVEEETTMLLENDSPSETIPEQTLLPDEFVSCLGIVNEELPSINTYFPQNILESH
FNRISLLEK
(SEQ ID NO: 4)

The present disclosure provides antagonists of IL-12Rβ1 and IL-23R signaling and their uses in the treatment of inflammatory diseases and autoimmune diseases. The IL-12Rβ1 antagonists of the present disclosure, including the neutralizing anti-IL-12Rβ1 designer cytokine antagonists of the present disclosure, can be used to block, inhibit, reduce, antagonize or neutralize the activity of either IL-12 or IL-23, or both IL-12 and IL-23 in the treatment of inflammation and inflammatory diseases such as multiple sclerosis, cancer (particularly as characterized by the expression of IL-17 and/or IL-23), psoriasis, psoriatic arthritis, rheumatoid arthritis, autoimmune ocular diseases, endotoxemia, IBS, and inflammatory bowel disease (IBD), colitis, asthma, allograft rejection, immune mediated renal diseases, hepatobiliary diseases, atherosclerosis, promotion of tumor growth, or degenerative joint disease, atherosclerosis, diabetes mellitus, and other inflammatory conditions disclosed herein.

The IL-23R antagonists described herein, including neutralizing anti-IL-23R designer cytokine antagonists, can be used to block, inhibit, reduce, antagonize or neutralize the activity of IL-23 in the treatment of inflammation and inflammatory diseases such as multiple sclerosis, cancer (particularly as characterized by the expression of IL-17 and/or IL-23), psoriasis, psoriatic arthritis, rheumatoid arthritis, autoimmune ocular diseases, endotoxemia, IBS, and inflammatory bowel disease (IBD), colitis, asthma, allograft rejection, immune mediated renal diseases, hepatobiliary diseases, atherosclerosis, diabetes mellitus, promotion of tumor growth, or degenerative joint disease, atherosclerosis, and other inflammatory conditions disclosed herein.

The present disclosure also provides antibodies that bind specifically to the immunomodulatory polypeptides described herein. In specific embodiments, the antibodies bind to the modified IL-23 polypeptides described herein but bind with lower specificity, or do not substantially bind, to wild-type IL-23 polypeptides.

The present disclosure provides isolated polypeptides that bind IL-12Rβ1, including an isolated polypeptide of SEQ ID NO:1 wherein one or more of amino acids within 130-152 and/or 29-47 of SEQ ID NO:1 are mutated to any other amino acid or deleted. More specifically, the present disclosure provides polypeptides that bind to IL-12Rβ1 and inhibit the production of an inflammatory mediator in a cell expressing IL-12Rβ1.

The present disclosure also provides isolated polypeptides having at least 80, 85, 88, 89, 90, 91, 92, 93, 94, 95, 96, 97, 98, or 99% identity to the polypeptide of SEQ ID NO:1, wherein one or more amino acids 130-152 and/or 29-47 of SEQ ID NO:1 are mutated to any other amino acid or deleted. Moreover, the present disclosure also provides isolated polypeptides as disclosed above that bind to, block, inhibit, reduce, antagonize or neutralize the activity of IL-12Rβ1. Further, the present disclosure also provides isolated polypeptides that are capable of interfering with the signaling, of IL-12 or IL-23, either singly or in combination.

The present disclosure also provides isolated polypeptides of at least 50, 60, 70, 80, 90, 100, 110, 120, 130, 140, 150, 160, 165, 166, 167, 168, or 169 amino acids in length, having at least 80, 85, 88, 89, 90, 91, 92, 93, 94, 95, 96, 97, 98, or 99% identity to the polypeptide of SEQ ID NO:1, wherein one or more amino acids 130-152 and/or 29-47 are mutated to any other amino acid or deleted.

The present disclosure also provides isolated polypeptides capable of binding the IL-23 p40 subunit.

Preferred embodiments of the invention include binding peptides, proteins, and any fragments or permutations thereof that bind to IL-12Rβ1 referred to interchangeably as “IL-12Rβ1 antagonists”, “antagonists of IL-12Rβ1”, “IL-12/IL-23 antagonists”, “IL-12 antagonists”, “IL-23 antagonists”, “IL-12Rβ1 neutralizing entities”, “IL-12Rβ1 designer cytokine antagonists”, “IL-23 designer cytokine antagonists”, “dominant-negative antagonists”, etc.). Specifically, such binding peptides or proteins are capable of specifically binding to human IL-12Rβ1 and/or to the human IL-23 p40 subunit. Further, these binding peptides or proteins are capable of modulating biological activities associated with either or both IL-12 and IL-23, and thus are useful in the treatment of various diseases and pathological conditions such as inflammation and immune-related diseases.

A preferred embodiment of the invention includes a designer cytokine antagonist of IL-12Rβ1 derived from the mutation or deletion of site 3 binding determinants of IL-23 p19. Specifically, the preferred embodiment comprises an isolated polypeptide of SEQ ID: 1 wherein one or more of amino acids 130-152 and/or 29-47 are mutated to any other amino acid or deleted. For example, the polypeptide can include between one and twenty, one and ten, one and five, two and seven, or two and five such mutations, and/or no greater than ten, five, four, two or one deletions, e.g., no deletions. The polypeptide can have, e.g., one, two, three, four, five, or six such mutations.

The polypeptide can be bound to p40 to form an IL-23 complex. For example, the complex has less than 20, 10, 5, 2, 1, 0.1, or 0.01% of the activity of a wildtype complex comprising SEQ ID NO:1 bound to an IL-23 p40 subunit. The activity can be a signaling activity, e.g., in a cell-based assay. The IL-23 complex can competitively inhibits a wildtype complex from IL-12Rβ1 mediated signaling, e.g., IL-23 and/or IL-12 mediated signaling, e.g., in vitro or in vivo. The IL-23 complex can inhibit the production of an inflammatory mediator, e.g., a mediator described herein.

For example, the IL-23 complex binds to IL-12Rβ1 with an affinity comparable to a wildtype complex, e.g., an affinity not more than 50, 10, or 5 fold weaker than a wildtype complex comprising SEQ ID NO:1 bound to an IL-23 p40 subunit. Conversely the complex does not detectably bind to IL-23R, in the presence or absence of IL-12Rβ1, or binds to IL-23R with at least a 10, 50, 500, or 1000-fold weaker binding affinity than the wildtype complex, e.g., in the presence or absence of IL-12Rβ1.

Another embodiment of the invention includes an isolated polypeptide comprising IL-12Rβ1 binding determinants of IL-23 wherein the binding determinants are selected from amino acids 10-27 or 101-117 of SEQ ID NO: 1.

In still another embodiment, the invention concerns an isolated polynucleotide that encodes a polypeptide of the present disclosure, wherein said polypeptide is capable of binding to IL-12Rβ1 and reducing its signaling capability.

The present disclosure provides isolated polypeptides that bind IL-23R, including an isolated polypeptide of SEQ ID:1 wherein one or more of amino acids 10-27 and 101-117 are mutated to any other amino acid or deleted. More specifically, the present disclosure provides polypeptides that bind to IL-23R and inhibit the production of an inflammatory mediator in a cell expressing IL-23R.

The present disclosure also provides isolated polypeptides having at least 80, 85, 88, 89, 90, 91, 92, 93, 94, 95, 96, 97, 98, or 99% identity to the polypeptide of SEQ ID NO:1, wherein one or more amino acids 10-27 and 101-117 are mutated to any other amino acid or deleted. Moreover, the present disclosure also provides isolated polypeptides as disclosed above that bind to, block, inhibit, reduce, antagonize or neutralize the activity of IL-23R. Further, the present disclosure also provides isolated polypeptides that are capable of interfering with the signaling of IL-23.

The present disclosure also provides isolated polypeptides of at least 50, 60, 70, 80, 90, 100, 110, 120, 130, 140, 150, 160, 165, 166, 167, 168, or 169 amino acids in length, having at least 80, 85, 88, 89, 90, 91, 92, 93, 94, 95, 96, 97, 98, or 99% identity to the polypeptide of SEQ ID NO:1, wherein one or more amino acids 10-27 or 101-117 are mutated to any other amino acid or deleted.

The present disclosure also provides isolated polypeptides capable of binding the IL-23 p40 subunit.

The present disclosure also provides antibodies that bind specifically to the immunomodulatory polypeptides described herein. In specific embodiments, the antibodies bind to the modified IL-23 polypeptides described herein but bind with lower specificity, or do not substantially bind, to wild-type IL-23 polypeptides. Preferred embodiments include binding peptides, proteins, and any fragments or permutations thereof that bind to IL-23R referred to interchangeably as “IL-23R antagonists”, “antagonists of IL-23R”, “IL-23 antagonists”, “IL-23R neutralizing entities”, “IL-23R designer cytokine antagonists”, “IL-23 designer cytokine antagonists”, etc.). Specifically, such binding peptides or proteins are capable of specifically binding to human IL-23R and/or to the IL-23 p40 subunit. Further, these binding peptides or proteins are capable of modulating biological activities associated with IL-23, and thus are useful in the treatment of various diseases and pathological conditions such as inflammation and immune-related diseases.

A preferred embodiment of the disclosure includes a designer cytokine antagonist of IL-23R derived from the mutation or deletion of site 2 binding determinants of IL-23 p19. Specifically, the preferred embodiment contains an isolated polypeptide of SEQ ID:1 wherein one or more of amino acids 10-27 or 101-117 are mutated to any other amino acid or deleted. For example, the polypeptide can include between one and twenty, one and ten, one and five, two and seven, or two and five such mutations, and/or no greater than ten, five, four, two or one deletions, e.g., no deletions. The polypeptide can have, e.g., one, two, three, four, five, or six such mutations.

The polypeptide can be bound to p40 to form an IL-23 complex. For example, the complex has less than 20, 10, 5, 2, 1, 0.1, or 0.01% of the activity of a wildtype complex comprising SEQ ID NO:1 bound to an IL-23 p40 subunit. The activity can be a signaling activity, e.g., in a cell-based assay. The IL-23 complex can competitively inhibits a wildtype complex from IL-23R mediated signaling, e.g., IL-23 mediated signaling, e.g., in vitro or in vivo. The IL-23 complex can inhibit the production of an inflammatory mediator, e.g., a mediator described herein.

For example, the IL-23 complex binds to IL-23R with an affinity comparable to a wildtype complex, e.g., an affinity not more than 50, 10, or 5 fold weaker than a wildtype complex comprising SEQ ID NO:1 bound to an IL-23 p40 subunit. Conversely the complex does not detectably bind to IL-12Rβ1, e.g., in the presence or absence of IL-23R, or binds to IL-12Rβ1 with at least a 10, 50, 500, or 1000-fold weaker binding affinity than the wildtype complex, e.g., in the presence or absence of IL-23R.

Another embodiment of the disclosure includes an isolated polypeptide comprising IL-23R binding determinants of IL-23 wherein the binding determinants are selected from amino acids 130-152 or 29-147 of SEQ ID NO:1.

In still another embodiment, the disclosure concerns an isolated polynucleotide that encodes a polypeptide of the present disclosure, wherein said polypeptide is capable of binding to IL-23R and reducing its signaling capability.

The present disclosure also provides fusion proteins, comprising an antagonist of the present disclosure and an immunoglobulin moiety. In such fusion proteins, the immunoglobulin moiety may be an immunoglobulin heavy chain constant region, such as a human F_{c} fragment. The present disclosure further includes isolated nucleic acid molecules that encode such fusion proteins.

The present disclosure also provides protein conjugates comprising an antagonist of the present disclosure conjugated to a polymer of polyethylene glycol.

The present disclosure further includes pharmaceutical compositions, comprising a pharmaceutically acceptable carrier and an IL-12Rβ1 antagonist polypeptide described herein.

In another aspect, the invention concerns a method for the treatment of an inflammatory disease characterized by elevated expression of IL-17 and/or IL-23 and/or IFN-γ in a mammalian subject, comprising administering to the subject an effective amount of an antagonist of IL-12 and IL-23 signaling, singly or in combination.

In yet another embodiment, the invention concerns a method for inhibiting the production of an inflammatory mediator in a mammalian cell by treating the cell or its media with an antagonist of IL-12Rβ1.

In another aspect, the invention concerns a method for the treatment of an inflammatory disease characterized by elevated expression of IL-17 and/or IL-23 and/or IFN-γ in a mammalian subject, comprising administering to the subject an effective amount of an antagonist of IL-12 and IL-23 signaling, singly or in combination.

Typical methods of the invention include methods to treat pathological conditions or diseases in mammals associated with or resulting from increased or enhanced IL-17 and/or IL-23 and/or IFN-γ expression and/or activity. In the methods of treatment, the antagonists of the present disclosure may be administered which preferably reduce the respective receptor activation. The methods contemplate the use of an antagonist of IL-12Rβ1 that reduces signaling by blocking IL-12Rβ1 association with IL-12Rβ2, IL-23R, and/or both.

Antagonists of the present disclosure (that is, antagonists of IL-12Rβ1) are also useful to prepare medicines and medicaments for the treatment of immune-related and inflammatory diseases, including for example, systemic lupus erythematosis, arthritis, rheumatoid arthritis, osteoarthritis, psoriasis, demyelinating diseases of the central and peripheral nervous systems such as multiple sclerosis, idiopathic demyelinating polyneuropathy or Guillain-Barre syndrome, inflammatory bowel disease, colitis, ulcerative colitis, Crohn's disease, gluten-sensitive enteropathy, autoimmune ocular diseases, cancer, neoplastic diseases, atherosclerosis, and angiogenesis.

In a specific aspect, such medicines and medicaments comprise a therapeutically effective amount of an IL-12Rβ1 designer cytokine antagonist with a pharmaceutically acceptable carrier. Preferably, the admixture is sterile.

In yet another embodiment, the invention concerns a method for inhibiting IL-17 production and/or maintenance by treating the T cells with an antagonist of IL-12Rβ1.

In a still further embodiment, the invention provides a method of decreasing the activity of T-lymphocytes in a mammal comprising administering to said mammal an IL-12Rβ1 antagonist, such as an IL-12Rβ1 designer cytokine antagonist, wherein the activity of T-lymphocytes in the mammal is decreased.

In a still further embodiment, the invention provides a method of decreasing the proliferation of T-lymphocytes in a mammal comprising administering to said mammal an IL-12Rβ1 antagonist, such as an IL-12Rβ1 designer cytokine antagonist, wherein the proliferation of T-lymphocytes in the mammal is decreased.

Processes for producing the same are also herein described, wherein those processes comprise culturing a host cell comprising a vector which comprises the appropriate encoding nucleic acid molecule under conditions suitable for expression of said antibody and recovering said antibody from the cell culture.

Production of Antibodies

Antibodies to the p19 subunit of IL-23 can be obtained, for example, by expressing p19 using an expression vector. Particularly useful anti-polypeptide antibodies “bind specifically” with the desired polypeptides. Antibodies are considered to be specifically binding if the antibodies exhibit at least one of the following two properties: (1) antibodies bind to the desired polypeptide(s) of with a threshold level of binding activity, and/or (2) antibodies do not significantly cross-react with unrelated polypeptides.

With regard to the first characteristic, antibodies specifically bind if they bind to a desired polypeptide, peptide or epitope with a binding affinity 10⁶ M or greater, preferably 10⁷ M or greater, more preferably 10⁸ M or greater, and most preferably 10⁹ M or greater. The binding affinity of an antibody can be readily determined by one of ordinary skill in the art, for example, by surface Plasmon resonance, radioimmunoassay, and Scatchard analysis (Scatchard, Ann. NY Acad. Sci. 51:660 (1949)). With regard to the second characteristic, antibodies do not significantly cross-react with unrelated polypeptide molecules, for example other cytokines.

Anti-polypeptide antibodies can be produced using antigenic epitope-bearing peptides and polypeptides. Antigenic epitope-bearing peptides and polypeptides of the present disclosure contain a sequence of at least 9, 10, 11, 12, 13, 14, or between 15 to about 30 amino acids contained within SEQ ID NO: 1 or another amino acid sequence disclosed herein. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies. It is desirable that the amino acid sequence of the epitope-bearing peptide is selected to provide substantial solubility in aqueous solvents (that is, the sequence includes relatively hydrophilic residues, while hydrophobic residues are preferably avoided). Moreover, amino acid sequences containing proline residues can also be desirable for antibody production.

Any suitable method known in the art may be used to determine the residues bound by an antibody. Such methods include hydrogen-deuterium exchange, site-directed mutagenesis, mass spectrometry, NMR and X-ray crystallography. The specific region or epitope of p19 that is bound can be identified by any suitable epitope mapping method. Examples of such methods include screening peptides of varying lengths derived from p19 for binding; the smallest peptide fragments that can specifically bind to the antibody contain the epitope recognized by the antibody. Peptides that bind the antibody can be identified by, for example, mass spectrometry analysis. In another example, NMR spectroscopy herein can be used to identify residues which interact with an antibody of the present invention. In still another example, the antibody is bound to mutated forms of p19. Mutations that perturb binding but not folding and expression are likely part of the epitope bound by the antibody.

Polyclonal antibodies to a polypeptide can be prepared using methods well-known to those of skill in the art. See, for example, Green et al., “Production of Polyclonal Antisera,” in Immunochemical Protocols (Manson, ed.) (Humana Press 1992), and Williams et al., “Expression of foreign proteins in E. coli using plasmid vectors and purification of specific polyclonal antibodies,” in DNA Cloning 2: Expression Systems, 2^{nd} Edition, Glover et al. (eds.) (Oxford University Press 1995). The immunogenicity of a polypeptide can be increased through the use of an adjuvant, such as alum (aluminum hydroxide) or Freund's complete or incomplete adjuvant. Polypeptides useful for immunization also include fusion polypeptides, such as fusions of polypeptides derived from SEQ ID NO: 1 with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an anti-polypeptide antibody can also be derived from a subhuman primate antibody. General techniques for raising diagnostically and therapeutically useful antibodies in baboons can be found, for example, in Goldenberg et al., patent publication No. WO 91/11465, and in Losman et al., Int. J. Cancer 46:310 (1990).

Monoclonal antibodies can be generated. Rodent monoclonal antibodies to specific antigens may be obtained by methods known to those skilled in the art (See, for example, Kohler et al., Nature 256:495 (1975); Coligan et al. (eds.), Current Protocols in Immunology (John Wiley & Sons 1991); Picksley et al., “Production of monoclonal antibodies against proteins expressed in E. coli,” in DNA Cloning 2: Expression Systems, 2^{nd} Edition, Glover et al. (eds.) (Oxford University Press 1995)).

Briefly, monoclonal antibodies can be obtained by injecting mice with a composition including the polypeptide, verifying the presence of antibody production by removing a serum sample, removing the spleen to obtain B-lymphocytes, fusing the B-lymphocytes with myeloma cells to produce hybridomas, cloning the hybridomas, selecting positive clones that produce antibodies to the antigen, culturing the clones that produce antibodies to the antigen, and isolating the antibodies from the hybridoma cultures.

Human antibodies to the polypeptide can also be derived. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan; Baines et al., “Purification of Immunoglobulin G (IgG),” in Methods in Molecular Biology, (The Humana Press, Inc. 1992)).

For particular uses, it can be desirable to prepare fragments of antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5 S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647; Nisonoff et al., Arch Biochem. Biophys. 89:230 (1960); Porter, Biochem. J. 73:119 (1959); Edelman et al., in Methods in Enzymology (Academic Press 1967); and by Coligan.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., (1972) PNAS. 69:2659. Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains, which are connected by a peptide linker. These single-chain antigen binding proteins (scFv) can be prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology (1991) (also see, Bird et al., Science 242:423 (1988); Ladner et al., U.S. Pat. No. 4,946,778; Pack et al., Bio/Technology 11:1271 (1993); and Sandhu, supra).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), (Cambridge University Press 1995); and Ward et al., “Genetic Manipulation and Expression of Antibodies,” in Monoclonal Antibodies: Principles and Applications, Birch et al., (eds.), (Wiley-Liss, Inc. 1995)).

An anti-polypeptide antibody can be a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986); Carter et al., Proc. Nat'l Acad. Sci. USA 89:4285 (1992); Sandhu, Crit. Rev. Biotech. 12:437 (1992); Singer et al., Immun. 150:2844 (1993); Sudhir (ed.), Antibody Engineering Protocols (Humana Press, Inc. 1995); Kelley, “Engineering Therapeutic Antibodies,” in Protein Engineering: Principles and Practice, Cleland et al. (eds.) (John Wiley & Sons, Inc. 1996); and by Queen et al., U.S. Pat. No. 5,693,762.

Polyclonal anti-idiotype antibodies can be prepared by immunizing animals with anti-polypeptide antibodies or antibody fragments, using standard techniques. See, for example, Green et al., “Production of Polyclonal Antisera,” in Methods In Molecular Biology: Immunochemical Protocols, Manson (ed.), (Humana Press 1992); Coligan. Alternatively, monoclonal anti-idiotype antibodies can be prepared using anti-polypeptide antibodies or antibody fragments as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677; and Varthakavi and Minocha, J. Gen. Virol. 77:1875 (1996).

A variety of assays known to those skilled in the art can be utilized to detect antibodies which specifically bind to a polypeptide. Exemplary assays are described in detail in Antibodies: A Laboratory Manual, Harlow and Lane (Eds.), Cold Spring Harbor Laboratory Press, 1988.

Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay.

For some applications (for example, certain therapeutic applications) it is preferred to use neutralizing antibodies. As used herein, the term “neutralizing antibody” denotes an antibody that inhibits at least 50%, 55%, 60%, 65%, 70%, 75%, 80%, 85%, 90%, 95% or more of the biological activity of the cognate antigen when the antibody is added at a molar access. Those of skill in the art will recognize that greater neutralizing activity is sometimes desirable, and antibodies that provide 50% inhibition at a 100-fold or 10-fold molar access may be advantageously employed.

F_{c} Fusion Polypeptides

A polypeptide disclosed herein, e.g. a p19 polypeptide, can be associated with a heterologous domain, such as the F_{c} region of an immunoglobulin. For example, the p19 polypeptide and the F_{c} region can be components of the same polypeptide chain, and can for example be joined by a linker. An exemplary F_{c} region is a human IgG₁. The heterologous polypeptide can include all or a portion of the CH2 domain, the CH3 domain, and/or a hinge region, of an immunoglobulin.

Fragments of an F_{c} region can also be used, as can F_{c} muteins. For example, certain residues within the hinge region of an F_{c} region are critical for high affinity binding to F_{c}γRI. Canfield and Morrison (1991) J. Exp. Med. 173:1483) reported that Leu234 and Leu235 are critical to high affinity binding of IgG₃ to F_{c}γRI present on U937 cells. Similar results were obtained by Lund et al. (1991) J. Immunol. 147:2657. Such mutations, alone or in combination, can be made in an IgG₁ F_{c} region to decrease the affinity of IgG₁ for FcR. Other F_{c} muteins that effect F_{c} binding, Antibody-Dependent Cell mediated Cytotoxicity (ADCC) and Complement Dependent Cytotoxicity (CDC) are described in Shields et al. (2001) J. Biol. Chem. 276(9):6591 and Lazar et al. U.S. Patent Application Publication No. 2004/0132101.

When immunoglobulin partners are used as conjugates, conjugate partners can be linked to any region of an immunoglobulin, including at the N- or C-termini, or some residue in-between the termini. A variety of linkers can be used to covalently link conjugate partners to an immunoglobulin. Linkers are known in the art; for example homo-or hetero bifunctional linkers. A number of strategies can be used to covalently link molecules together. These include, but are not limited to polypeptide linkages between N- and C-termini of proteins or protein domains, linkage via disulfide bonds, and linkage via chemical cross-linking reagents. A peptide linker may include the amino acids: Gly, Ser, Ala, or Thr. Such a linker should possess a length that is adequate to link two molecules in such a way that they assume the correct conformation relative to one another so that they retain the desired activity. Suitable lengths for this purpose include at least one and not more than 50 amino acid residues.

An exemplary peptide linker has the amino acid sequence: GGSGG SGGGG SGGGG S (SEQ ID NO:5). An exemplary F_{c} moiety is a human y4 chain, which is stable in solution and has little or no complement activating activity.

Amino Acid Modifications

Polypeptides can be modified in a variety of ways including substitution, deletion, or addition. A substitution entails the replacement of one amino acid for another. Such replacements can be made using any one of the twenty amino acids directly encoded by the genetic code: alanine, arginine, asparagine, aspartic acid, cysteine, glutamic acid, glutamine, glycine, histidine, isoleucine, leucine, lysine, methionine, phenylalanine, proline, serine, threonine, tryptophan tyrosine, and valine. In addition, amino acids of a polypeptide can be replaced using amino acids not directly encoded by the genetic code including: selenocysteine, pyrrolysine, p-nitrophenylalanine, p-sulfotyrosine, p-carboxyphenylalanine, o-nitrophenylalanine, m-nitrophenylalanine, p-boronyl Phe; o-boronyl Phe; m-boronyl Phe; p-amino Phe, o-amino Phe, m-amino Phe, p-acyl Phe, o-acyl Phe, m-acyl Phe, p-OMe Phe, o-OMe Phe, m-OMe Phe, p-sulfo Phe, o-sulfo Phe, m-sulfo Phe, 5-nitro His, 3-nitro Tyr, 2-nitro Tyr, nitro substituted Leu, nitro substituted His, nitro substituted Ile, nitro substituted Trp, 2-nitro Trp, 4-nitro Trp, 5-nitro Trp, 6-nitro Trp, 7-nitro Trp, 3-aminotyrosine, 2-aminotyrosine, O-sulfotyrosine, 2-sulfooxyphenylalanine, 3-sulfooxyoxyphenylalanine, p-carboxyphenylalanine, o-carboxyphenyalanine, and m-carboxyphenylalanine.

Conservative amino acid substitutions can frequently be made in a protein without altering either the conformation or the function of the protein. Typical conservative changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, for example, “Biochemistry” 2^{nd} ed. Lubert Stryer ed (Stanford University); Henikoff et al., (1992) PNAS 89 10915; Lei et al., (1995) J Biol Chem 270(20):11882).

Substitutions can be chosen based on their potential effect on (a) backbone structure in the vicinity of the substitution, for example, a sheet or helical conformation, (b) the charge or hydrophobicity of the molecule at the target site, or (c) the volume and branching of the side chain.

Amino acid residues can be classified based on side-chain properties: (1) aliphatic: met, val, leu, ile; (2) neutral hydrophilic: ser, thr; asn; gln; (3) acidic: asp, glu; (4) basic: his, lys, arg; (5) residues that affect backbone conformation: gly, pro; and (6) aromatic: trp, tyr, phe. Non-conservative substitutions can include substituting a member of one of these classes for a member of a different class or making a substitution not identified in the table below. Conservative substitutions can include substituting a member of one of these classes for another member of the same class. Generally mutations are not made to cys.

Exemplary conservative substitutions are described in the following table:

TABLE 3

| | Exemplary | Further Specific |
|---|---|---|
| Original | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; | leu |
| | phe; leu | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; leu; | ala |
| | norleucine | |

Exemplary mutations in p19 (with reference to the numbering in SEQ ID NO: 1) include the following with respect to a p19 polypeptide that is deficient in its ability to bind to the extracellular domain of IL-23R, but retains its ability to bind to the extracellular domain of IL-12Rβ1:

Ser130 can be mutated to another amino acid, e.g., alanine or an amino acid other than alanine. In one embodiment, Ser130 is mutated to an acidic or basic residue, or to an aliphatic or aromatic residue.

Leu131 can be mutated to another amino acid, e.g., alanine or an amino acid other than alanine. In one embodiment, Leu131 is mutated to an acidic or basic residue. In another embodiment, Leu131 is mutated to a neutral hydrophilic residue or to an aliphatic or aromatic residue.

Ser132 can be mutated to another amino acid, e.g., alanine or an amino acid other than alanine. In one embodiment, Ser132 is mutated to an acidic or basic residue, or to an aliphatic or aromatic residue.

Pro133 can be mutated to another amino acid, e.g., alanine or an amino acid other than alanine. In one embodiment, Pro133 is mutated to glycine. In another embodiment, Pro133 is mutated an acidic or basic residue, e.g., histidine. In still another embodiment, Pro133 is mutated to an aliphatic or aromatic residue.

Ser134 can be mutated to another amino acid, e.g., alanine or an amino acid other than alanine. In one embodiment, Ser134 is mutated to an acidic or basic residue, or to an aliphatic or aromatic residue.

Gln135 can be mutated to another amino acid, e.g., alanine or an amino acid other than alanine. In one embodiment, Gln135 is mutated to a basic residue, or to an aliphatic or aromatic residue.

Pro136 can be mutated to another amino acid, e.g., alanine or an amino acid other than alanine. In one embodiment, Pro136 is mutated to glycine. In another embodiment, Pro136 is mutated an acidic or basic residue, e.g., histidine. In still another embodiment, Pro136 is mutated to an aliphatic or aromatic residue.

Trp137 can be mutated to another amino acid, e.g., alanine or an amino acid other than alanine. In one embodiment, Trp137 is mutated to a basic or acidic residue, or to a neutral hydrophilic residue, or an aliphatic residue (e.g., val, leu, ile).

Gln138 can be mutated to another amino acid, e.g., alanine or an amino acid other than alanine. In one embodiment, Gln138 is mutated to a basic residue. In another embodiment, Gln138 is mutated to an aromatic residue, an acidic or an aliphatic residue.

Arg139 can be mutated to another amino acid, e.g., alanine or an amino acid other than alanine. In one embodiment, Arg139 is mutated to an acidic residue or a neutral hydrophilic residue. In another embodiment, Arg139 is mutated to an aromatic residue or to Lys.

Leu140 can be mutated to another amino acid, e.g., alanine or an amino acid other than alanine. In one embodiment, Leu140 is mutated to an acidic or basic residue or to a neutral hydrophilic residue. In another embodiment, Leu140 is mutated to another aliphatic residue or to an aromatic residue.

Leu141 can be mutated to another amino acid, e.g., alanine or an amino acid other than alanine. In one embodiment, Leu141 is mutated to an acidic or basic residue. In another embodiment, Leu141 is mutated to a neutral hydrophilic residue or to an aromatic residue.

Leu142 can be mutated to another amino acid, e.g., alanine or an amino acid other than alanine. In one embodiment, Leu142 is mutated to an acidic or basic residue. In another embodiment, Leu142 is mutated to a neutral hydrophilic residue or to an aromatic residue.

Arg143 can be mutated to another amino acid, e.g., alanine or an amino acid other than alanine. In one embodiment, Arg143 is mutated to Lys. In another embodiment, Arg143 is mutated to an aliphatic residue. In still another embodiment, Arg143 is not mutated.

Phe144 can be mutated to another amino acid, e.g., alanine or an amino acid other than alanine. In one embodiment, Phe144 is mutated to an acidic or basic residue, or to a neutral hydrophilic residue. In another embodiment, Phe144 is mutated to an aliphatic residue.

Lys145 can be mutated to another amino acid, e.g., alanine or an amino acid other than alanine. In one embodiment, Lys145 is mutated to an acidic residue. In another embodiment, Lys145 is mutated to a neutral hydrophilic residue or an aromatic residue. In still another embodiment, Lys145 is mutated to an aliphatic residue.

Ile146 can be mutated to another amino acid, e.g., alanine or an amino acid other than alanine. In one embodiment, Ile146 is mutated to another aliphatic residue. In still another embodiment, Ile146 is not mutated.

Leu147 can be mutated to another amino acid, e.g., alanine or an amino acid other than alanine. In one embodiment, Leu147 is mutated to another aliphatic residue. In still another embodiment, Leu147 is not mutated.

Arg148 can be mutated to another amino acid, e.g., alanine or an amino acid other than alanine. In one embodiment, Arg148 is mutated to an acidic residue or a neutral hydrophilic residue. In another embodiment, Arg148 is mutated to an aromatic residue or to Lys.

Ser149 can be mutated to another amino acid, e.g., alanine or an amino acid other than alanine. In one embodiment, Ser149 is mutated to an acidic or basic residue, or to an aliphatic or aromatic residue.

In one embodiment, a plurality of amino acid residues in the loop region from 130-136 are mutated, e.g., at least two or three residues. For example, Trp137 and at least two amino acids in the loop region from 130-136 are mutated. In one embodiment, an amino acid residue from the loop region is deleted, e.g., one, two, or three residues are deleted. In one embodiment, none of the amino acid residues in the loop region are deleted. In one embodiment, none of the amino acid residues from 130-152 are deleted. In particular, none of the amino acid residues in the helical region of residues 137-152 are deleted.

In one embodiment, at least Trp137 and Leu141 are mutated. In one embodiment, at least Trp137 and Lys145 are mutated. For example, Trp137, Leu141, and Lys145 are mutated. In another embodiment, Trp137, Gln138, and Leu141 are mutated.

In one embodiment, residues in the α-helix that includes residues 137-152 and that pack against the core of the protein are not mutated and identical to the naturally occurring residues. Exemplary residues that pack against the core include Arg143, Ile146, Leu147, and Leu150.

One or more residues selected from the group consisting of residues 29, 30, 31, 32, 33, 34, 35, 36, 37, 38, 39, 40, 41, 42, 43, 44, 45, 46 or 47 can be mutated or deleted. The residues can be mutated, e.g., to alanine or a non-conserved amino acid, e.g., individually or in groups, e.g., of 2, 3, 4, or 5 mutations, or more.

For example: His29 can be mutated to another amino acid, e.g., an uncharged amino acid, e.g., an aliphatic amino acid, or to Arg or Lys. Pro30 can be mutated to another amino acid, e.g., glycine, a charged amino acid, or an aliphatic amino acid. Leu31 can be mutated to another amino acid, e.g., a charged amino acid or an uncharged hydrophilic amino acid. Val32 can be mutated to another amino acid, e.g., a charged amino acid or an uncharged hydrophilic amino acid. Gly33 can be mutated to another amino acid, e.g., an aliphatic amino acid, a charged amino acid, or an uncharged hydrophilic amino acid. His34 can be mutated to another amino acid, e.g., an uncharged amino acid, e.g., an aliphatic amino acid, or to Arg or Lys. Met35 can be mutated to another amino acid, e.g., a charged amino acid or an uncharged hydrophilic amino acid. Asp36 can be mutated to another amino acid, e.g., an uncharged hydrophilic amino acid or a hydrophobic amino acid, e.g., an aliphatic amino acid or an aromatic amino acid, or to a basic amino acid. Leu37 can be mutated to another amino acid, e.g., to a charged amino acid or an uncharged hydrophilic amino acid. Arg38 can be mutated to another amino acid, e.g., an uncharged hydrophilic amino acid or a hydrophobic amino acid, e.g., an aliphatic amino acid or an aromatic amino acid, or to an acidic amino acid. One or more of Glu39, Glu40, Glu43, and Glu44 can be mutated to another amino acid, e.g., an uncharged hydrophilic amino acid or a hydrophobic amino acid, e.g., an aliphatic amino acid, or to a basic amino acid. Gly41 be mutated to another amino acid, e.g., an aliphatic amino acid, a charged amino acid, or an uncharged hydrophilic amino acid. One or more of Thr45, Thr46, and Asn47 can be mutated to another amino acid, e.g., a charged amino acid or a hydrophobic amino acid, e.g., an aliphatic or aromatic amino acid.

Exemplary mutations in p19 (with reference to the numbering in SEQ ID NO: 1) include the following with respect to a p19 polypeptide that is deficient in its ability to bind to the extracellular domain of IL-12Rβ1, but retains its ability to bind to IL-23R:

Ala10 can be mutated to another amino acid, e.g., an amino acid other than alanine. In one embodiment, Ala10 is mutated to a charged amino acid, e.g., a basic or acidic amino acid.

Trp11 can be mutated to another amino acid, e.g., alanine or an amino acid other than alanine. In one embodiment, Trp11 is mutated to an aliphatic amino acid or another aromatic amino acid. In on embodiment, Trp11 is not mutated.

Thr12 can be mutated to another amino acid, e.g., alanine or an amino acid other than alanine. In one embodiment, Thr12 is mutated to an acidic or basic residue, or to an aliphatic or aromatic residue.

One or more of Gln13, Gln15, and Gln16 can be mutated to another amino acid, e.g., alanine or an amino acid other than alanine. In one embodiment, one or more of Gln13, Gln15, and Gln16 are mutated to an acidic or basic residue, or to an aliphatic or aromatic residue.

Cys14 can be mutated to another amino acid, e.g., alanine or an amino acid other than alanine. In one embodiment, Cys14 is mutated to an aliphatic residue. In one embodiment, Cys14 is not mutated.

Leu17 can be mutated to another amino acid, e.g., alanine or an amino acid other than alanine. In one embodiment, Leu17 is mutated to an acidic or basic residue, or to an aromatic residue. In one embodiment, Leu17 is mutated to another aliphatic residue. In still another embodiment, Leu17 is not mutated.

Ser18 can be mutated to another amino acid, e.g., alanine or an amino acid other than alanine. In one embodiment, Ser18 is mutated to an aliphatic or aromatic residue, or to a charged amino acid.

Gln19 can be mutated to another amino acid, e.g., alanine or an amino acid other than alanine. In one embodiment, Gln19 is mutated to an acidic or basic residue, or to an aliphatic or aromatic residue.

Lys20 can be mutated to another amino acid, e.g., alanine or an amino acid other than alanine. In one embodiment, Lys20 is mutated to an acidic residue, or to an aliphatic or an aromatic residue.

Leu21 can be mutated to another amino acid, e.g., alanine or an amino acid other than alanine. In one embodiment, Leu21 is mutated to another aliphatic residue. In one embodiment, Leu21 is not mutated.

Cys22 can be mutated to another amino acid, e.g., alanine or an amino acid other than alanine. In one embodiment, Cys22 is mutated to a charged amino acid, e.g., an acidic or basic residue, or to an aromatic residue or an aliphatic residue. In one embodiment, Cys22 is mutated to Ser or Thr.

Thr23 can be mutated to another amino acid, e.g., alanine or an amino acid other than alanine. In one embodiment, Thr23 is mutated to an aliphatic or aromatic residue, or to a charged amino acid.

Leu24 can be mutated to another amino acid, e.g., alanine or an amino acid other than alanine. In one embodiment, Leu24 is mutated to a charged amino acid or an uncharged hydrophilic amino acid.

Ala25 can be mutated to another amino acid, e.g., an amino acid other than alanine. In one embodiment, Ala25 is mutated to a charged amino acid, e.g., a basic or acidic amino acid. In one embodiment, Ala25 is mutated to Ser, Thr, or a large aliphatic or an aromatic amino acid.

Trp26 can be mutated to another amino acid, e.g., an amino acid other than alanine. In one embodiment, Trp26 is mutated to a non-aromatic residue, e.g., to an aliphatic or basic residue. In one embodiment, Trp26 is mutated to another aromatic residue.

Ser27 can be mutated to another amino acid, e.g., alanine or an amino acid other than alanine. In one embodiment, Ser27 is mutated to an aliphatic or aromatic residue, or to a charged amino acid, e.g., a basic or acidic residue.

In one embodiment, residues in the a-helix that includes residues 10-27 and that pack against the core of the protein are not mutated and identical to the naturally occurring residues. Exemplary residues that pack against the core include Trp11, Cys14, Leu17, and Leu21.

Pro101 can be mutated to another amino acid, e.g., alanine or an amino acid other than alanine. In one embodiment, Pro101 is mutated to glycine or a charged amino acid.

Val102 can be mutated to another amino acid, e.g., alanine or an amino acid other than alanine. In one embodiment, Val102 is mutated to another aliphatic amino acid. In one embodiment, Val102 is not mutated.

Gly103 can be mutated to another amino acid, e.g., alanine or an amino acid other than alanine. In one embodiment, Gly103 is mutated to a charged amino acid.

Gln104 can be mutated to another amino acid, e.g., alanine or an amino acid other than alanine. In one embodiment, Gln104 is mutated to a charged amino acid, e.g., an acidic or basic amino acid, or to an aromatic amino acid or an aliphatic amino acid.

Leu105 can be mutated to another amino acid, e.g., alanine or an amino acid other than alanine. In one embodiment, Leu105 is mutated to another aliphatic amino acid. In one embodiment, Leu105 is not mutated.

His106 can be mutated to another amino acid, e.g., alanine or an amino acid other than alanine. In one embodiment, His106 is mutated to a charged amino acid, e.g., a basic amino acid (e.g., Arg or Lys) or an acidic amino acid, or to an aromatic amino acid or an aliphatic amino acid.

Ala107 can be mutated to another amino acid, e.g., an amino acid other than alanine. In one embodiment, Ala107 is mutated to a charged amino acid, e.g., a basic or acidic amino acid.

In one embodiment, Ala107 is mutated to Ser, Thr, or a large aliphatic or an aromatic amino acid.

Ser108 can be mutated to another amino acid, e.g., alanine or an amino acid other than alanine. In one embodiment, Ser108 is mutated to an aliphatic or aromatic residue, or to a charged amino acid, e.g., a basic or acidic residue.

Leu109 can be mutated to another amino acid, e.g., alanine or an amino acid other than alanine. In one embodiment, Leu109 is mutated to another aliphatic amino acid. In one embodiment, Leu105 is not mutated.

One or more of Leu110 and 112 can be mutated to another amino acid, e.g., alanine or an amino acid other than alanine. In one embodiment, one or more of Leu110 and Leu112 is mutated, e.g., to a charged amino acid, e.g., a basic or acidic amino acid, or to aromatic amino acid, or to another aliphatic amino acid.

Ser113 can be mutated to another amino acid, e.g., alanine or an amino acid other than alanine. In one embodiment, Ser113 is mutated to an aliphatic or aromatic residue, or to a charged amino acid, e.g., a basic or acidic residue. In one embodiment, Ser113 is not mutated.

Gln114 can be mutated to another amino acid, e.g., alanine or an amino acid other than alanine. In one embodiment, Gln114 is mutated to a charged amino acid, e.g., an acidic or basic amino acid, or to an aromatic amino acid or an aliphatic amino acid.

Leu115 can be mutated to another amino acid, e.g., alanine or an amino acid other than alanine. In one embodiment, Leu115 is mutated, e.g., to a charged amino acid, e.g., a basic or acidic amino acid, or to aromatic amino acid, or to another aliphatic amino acid.

Leu116 can be mutated to another amino acid, e.g., alanine or an amino acid other than alanine. In one embodiment, Leu116 is not mutated.

Gln117 can be mutated to another amino acid, e.g., alanine or an amino acid other than alanine. In one embodiment, Gln117 is mutated to a charged amino acid, e.g., an acidic or basic amino acid, or to an aromatic amino acid or an aliphatic amino acid.

In one embodiment, residues in the a-helix that includes residues 101-117 and that pack against the core of the protein are not mutated and identical to the naturally occurring residues. Exemplary residues that pack against the core include Val102, Leu105, Leu109, Ser113, and Leu116.

Polypeptide variants of SEQ ID NO:1 can be made, e.g., using methods known in the art such as site-directed mutagenesis, cassette mutagenesis and PCR mutagenesis. (Carter et al., (1986) Nucl. Acids Res., 13:4331; Zoller et al., (1985) Nucl. Acids Res., 10:6487; Wells et al., (1985) Gene, 34:315; Wells et al., (1986) Philos. Trans. R. Soc. London SerA, 317:415).

Methods of Use

The compositions described herein are useful in methods for treating or preventing a disease or disorder in a vertebrate subject. In one such method, the step of administering to the subject a composition containing one or more polypeptides is provided. As described herein, the composition is administered intravesicularly, topically, orally, rectally, ocularly, optically, nasally, or via inhalation.

Also provided are methods of using the modified polypeptides described herein to modulate the immune system of a vertebrate. A level of an inflammatory cytokine can be reduced upon the administration of a modified polypeptide in a mammalian subject, such as by administering to the subject a therapeutically effective amount of a composition comprising a modified IL-23. Exemplary inflammatory cytokines are IL-17A, IL-17F, IL-22, IL-23, IL-26, CCL20, CCR6, RORC, RORC2, RORγt, IL-1, IL-6, IL-23R, IL-21, IL-2, and TNF-α, which may be reduced individually or in combinations of two or more inflammatory cytokines. The level of inflammatory cytokine present in the blood and/or another tissue of the mammal is generally reduced. Modulation of the immune system also includes methods of increasing a level of an anti-inflammatory cytokine in a mammalian subject. For example, the anti-inflammatory cytokine is IL-10, IL-4, IL-11, IL-13, or TGF-β. Optionally, the level of the anti-inflammatory cytokine present in the blood of the mammal is increased.

The p19 proteins and antibodies described herein can be used to detect a IL-23 p40 subunit and/or the IL-12Rβ1 receptor in a sample. For example, the p19 protein or antibody can be labeled directly or indirectly with a moiety that is a label or produces a signal, e.g., an enzyme, a radiolabel, an epitope, or a fluorescent protein (such as green fluorescent protein). The p19 protein can be a fusion protein. The agent (e.g., the p19 protein or antibody) can be contacted to a sample or to cells to determine if the receptor is present in the sample or on the cells, e.g., using standard immunoblotting, immunofluorescence, enzyme immunoassay (EIA), radioimmunoassay (RIA), fluorescence energy transfer, Western blot, and other diagnostic and detection techniques.

The agent can also be labeled for in vivo detection and administered to a subject. The subject can be imaged, e.g., by NMR or other tomographic means. For example, the agent can be labeled with a radiolabel such as ¹³¹I, ¹¹¹In, ¹²³I, ^{99m}Tc, ³²P, ¹²⁵I, ³H, ¹⁴C, and ⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. The agent can be labeled with a contrast agent such as paramagnetic agents and ferromagnetic or superparamagnetic (which primarily alter T2 response).

The agent can also be used to purify cells which express the protein(s) to which it binds. For example, the agent can be coupled to an immobilized support (e.g., magnetic beads or a column matrix) and contacted to cells which may express the receptor. The support can be washed, e.g., with a physiological buffer, and the cells can be recovered from the support.

The agent can also be used to purify the protein(s) to which it binds. For example, samples containing the soluble receptor can be contacted to immobilized IL-23 p40 subunit and then, e.g., after washing, can be recovered from the immobilized IL-23 p40 subunit.

The compositions described herein may be used therapeutically or prophylactically. Cocktails of various different polypeptides can be used together to bind to and act upon one or multiple targets, for example, multiple cell types, at once. Successful treatment can be assessed by routine procedures familiar to a physician.

Formulations

One or more therapeutic agent, alone or in combination with one or more other therapeutic agents, can be formulated with a pharmaceutically acceptable carrier for administration to a subject. Pharmaceutical compositions can be substantially free of pyrogenic materials, substantially free of nucleic acids, and/or substantially free of cellular enzymes and components, such as polymerases, ribosomal proteins, and chaperone proteins.

A polypeptide or antibody described herein can be formulated according to standard methods for a biologic. See e.g., Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkens Publishers (1999) (ISBN: 0683305727): Kibbe (ed.). Handbook of Pharmaceutical Excipients, 3rd ed. (2000) (ISBN: 091733096X); Protein Formulation and Delivery, McNally and Hastedt (eds.), Informa Health Care (ISBN: 0849379490) (2007).

The term “pharmaceutically acceptable carrier” as used herein means one or more compatible solid or liquid filler, diluents or encapsulating substances which are suitable for administration to a subject. The components of the pharmaceutical compositions also are capable of being commingled with each other, in a manner such that there is no interaction, which would substantially impair the desired pharmaceutical efficiency. Such preparations may routinely contain pharmaceutically acceptable concentrations of salt, buffering agents, preservatives, compatible carriers, adjuvants and optionally other therapeutic ingredients.

The compositions described herein may be administered as a free base or as a pharmaceutically acceptable salt. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene sulphonic, and benzene sulphonic. Also, pharmaceutically acceptable salts can be prepared as alkaline metal or alkaline earth salts, such as sodium, potassium or calcium salts of the carboxylic acid group.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v).

Suitable preservatives include benzalkonium chloride (0.003-0.03% w/v); chlorobutanol (0.3-0.9% w/v); parabens (0.01-0.25% w/v) and thimerosal (0.004-0.02% w/v).

Suitable liquid or solid pharmaceutical preparation forms are, for example, aqueous or saline solutions for inhalation, microencapsulated, encochleated, coated onto microscopic gold particles, contained in liposomes (including pH-dependent release formulations), lipidoids, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of the compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990 and Langer and Tirrell, Nature, 2004 Apr. 1; 428(6982):487-92.

The compositions may conveniently be presented in unit dosage form and may be prepared by any of the methods well known in the art of pharmacy. In certain embodiments, the composition that is administered is in powder or particulate form rather than as a solution. Examples of particulate forms contemplated as part of the invention are provided in U.S. Patent Application Publication No. 2002/0128225. In some embodiments, the compositions are administered in aerosol form. In other embodiments, the compositions may be in powder form for constitution with a suitable vehicle, for example, sterile pyrogen-free water, before use.

In addition, the compositions described herein may be formulated as a depot preparation, time-release, delayed release or sustained release delivery system. Such systems can avoid repeated administrations of the compositions described herein, increasing convenience to the subject and the physician. Such long acting formulations may be formulated with suitable polymeric or hydrophobic materials (for example as an emulsion in an acceptable oil) or ion exchange resins, or as sparingly soluble derivatives, for example, as a sparingly soluble salt. Many types of release delivery systems are available and known to those of ordinary skill in the art. They include polymer based systems such as polylactic and polyglycolic acid, beta-glucan particles, polyanhydrides and polycaprolactone; nonpolymer systems that are lipids including sterols such as cholesterol, cholesterol esters and fatty acids, neutral fats such as mono-, di- and triglycerides or lipidoids; hydrogel release systems; silastic systems; peptide based systems; wax coatings, compressed tablets using conventional binders and excipients, partially fused implants and the like. Specific examples include, but are not limited to: (a) erosional systems in which the agent is contained in a form within a matrix, found in U.S. Pat. No. 4,452,775 (Kent); U.S. Pat. No. 4,667,014 (Nestor et al.); and U.S. Pat. No. 4,748,034 and U.S. Pat. No. 5,239,660 (Leonard) and (b) diffusional systems in which an agent permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, nonbioerodable/non-biodegradable polymers. Such polymers have been described in great detail in the prior art and include, but are not limited to: beta-glucan particles, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate. In one embodiment the slow release polymer is a block copolymer, such as poly(ethylene glycol) (PEG)/poly(lactic-co-glycolic acid) (PLGA) block copolymer.

Examples of non-biodegradable polymers include ethylene vinyl acetate, poly(meth) acrylic acid, polyamides, copolymers and mixtures thereof.

Examples of biodegradable polymers include synthetic polymers, for example, beta-glucan particles, polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. Preferred polymers are polyesters, polyanhydrides, polystyrenes and blends thereof.

Effective amounts of the compositions described herein are administered to a subject in need of such treatment. Effective amounts are those amounts, which will result in a desired improvement in the condition, disease or disorder or symptoms of the condition, disease or disorder.

Effective doses range from 1 ng/kg to 100 mg/kg body weight, or from 100 ng/kg to 50 mg/kg body weight, or from 1 μg/kg to 10 mg/kg body weight, depending upon the mode of administration. Alternatively, effective doses can range from 3 micrograms to 14 milligrams per 4 square centimeter area of cells. The absolute amount will depend upon a variety of factors (including whether the administration is in conjunction with other methods of treatment, the number of doses and individual patient parameters including age, physical condition, size and weight) and can be determined with routine experimentation. It is preferred, generally, that a maximum dose be used, that is, the highest safe dose according to sound medical judgment.

The time between the delivery of the various active agents can be defined rationally by first principles of the kinetics, delivery, release, agent pharmacodynamics, agent pharmacokinetics, or any combination thereof. Alternatively, the time between the delivery of the various agents can be defined empirically by experiments to define when a maximal effect can be achieved.

Mode of Administration

The mode of administration may be any medically acceptable mode including oral administration, sublingual administration, intranasal administration, intratracheal administration, inhalation, ocular administration, topical administration, transdermal administration, intradermal administration, rectal administration, vaginal administration, subcutaneous administration, intravenous administration, intramuscular administration, intraperitoneal administration, intrasternal, administration, or via transmucosal administration. In addition, modes of administration may be via an extracorporeal device and/or tissue-penetrating electro-magnetic device.

The particular mode selected will depend upon the particular active agents selected, the desired results, the particular condition being treated and the dosage required for therapeutic efficacy. The methods described herein, generally speaking, may be practiced using any mode of administration that is medically acceptable, for example, any mode that produces effective levels of inflammatory response alteration without causing clinically unacceptable adverse effects.

The compositions can be provided in different vessels, vehicles or formulations depending upon the disorder and mode of administration. For example, for oral application, the compositions can be administered as sublingual tablets, gums, mouth washes, toothpaste, candy, gels, films, etc.; for ocular application, as eye drops in eye droppers, eye ointments, eye gels, eye packs, as a coating on a contact lens or an intraocular lens, in contacts lens storage or cleansing solutions, etc.; for topical application, as lotions, ointments, gels, creams, sprays, tissues, swabs, wipes, etc.; for vaginal or rectal application, as an ointment, a tampon, a suppository, a mucoadhesive formulation, etc.

The compositions, may be administered by injection, e.g., by bolus injection or continuous infusion, via intravenous, subcutaneous, intramuscular, intraperitoneal, intrasternal routes. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

For oral administration, the compositions can be formulated readily by combining the compositions with pharmaceutically acceptable carriers well known in the art. Such carriers enable the compositions to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

One suitable oral form is a sublingual tablet. A sublingual tablet delivers the composition to the sublingual mucosa. As used herein, “tablet” refers to pharmaceutical dosage forms prepared by compressing or molding. Sublingual tablets are small and flat, for placement under the tongue and designed for rapid, almost instantaneous disintegration and release the composition to the sublingual mucosa, for example, within five minutes.

Oral formulations can also be in liquid form. The liquid can be administered as a spray or drops to the entire oral cavity including select regions such as the sublingual area. The sprays and drops of the present disclosure can be administered by means of standard spray bottles or dropper bottles adapted for oral or sublingual administration. The liquid formulation is preferably held in a spray bottle, fine nebulizer, or aerosol mist container, for ease of administration to the oral cavity. Liquid formulations may be held in a dropper or spray bottle calibrated to deliver a predetermined amount of the composition to the oral cavity. Bottles with calibrated sprays or droppers are known in the art. Such formulations can also be used in nasal administration.

The compositions can also be formulated as oral gels. As an example, the composition may be administered in a mucosally adherent, non-water soluble gel. The gel is made from at least one water-insoluble alkyl cellulose or hydroxyalkyl cellulose, a volatile nonaqueous solvent, and the composition. Although a bioadhesive polymer may be added, it is not essential. Once the gel is contacted to a mucosal surface, it forms an adhesive film due primarily to the evaporation of the volatile or non-aqueous solvent. The ability of the gel to remain at a mucosal surface is related to its filmy consistency and the presence of non-soluble components. The gel can be applied to the mucosal surface by spraying, dipping, or direct application by finger or swab.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the compositions may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of for example, gelatin for use in an inhaler or insufflator can be formulated containing a powder mix of the compositions and a suitable powder base such as lactose or starch. Medical devices for the inhalation of therapeutics are known in the art. In some embodiments the medical device is an inhaler. In other embodiments the medical device is a metered dose inhaler, diskhaler, Turbuhaler, diskus or a spacer. In certain of these embodiments the inhaler is a Spinhaler (Rhone-Poulenc Rorer, West Mailing, Kent). Other medical devices are known in the art and include Inhale/Pfizer, Mannkind/Glaxo and Advanced Inhalation Research/Alkermes.

The compositions can also be formulated in rectal or vaginal compositions such as suppositories or retention enemas, for example containing conventional suppository bases such as cocoa butter or other glycerides.

The entire disclosure of each of the patent documents and scientific articles referred to herein, and those patent documents and scientific articles cited thereby, is expressly incorporated by reference herein for all purposes.

### EXAMPLES

### Example 1

### IL-23 Expression and Crystallization

IL-23 was expressed by co-infection of insect HIFIVE™ cells with recombinant baculoviruses carrying cDNAs for p19 and p40. The complex purified by gel filtration as a single peak of disulfide-linked dimer, confirming the integrity of the IL-23 heterodimer as shown for IL-12 (Yoon, C., Johnston, S. C., Tang, I, Stahl, M., Tobin, J. F. & Somers, W. S. (2000). Charged residues dominate a unique interlocking topography in the heterodimeric cytokine interleukin-12. Embo J 19:3530-41.). IL-23 was crystallized and the structure determined to a resolution of 2.3 Å by molecular replacement using the coordinates of p40 (Yoon, C., et al.), and p19 was then built from the partial phases in order to eliminate model bias that would be introduced by using p35 as a starting model. During the expression, Asn-linked glycans were deglycosylated with EndoH in order to debulk the complex of carbohydrate moieties, and the surface lysines were methylated in order to grow large, well-ordered crystals. The deglycosylated and methylated IL-23 behaved identically to unmodified IL-23 during purification, while crystal diffraction was markedly improved (˜4.5 Å to 2.3 Å).

### Example 2

### IL-23 Overall Structure

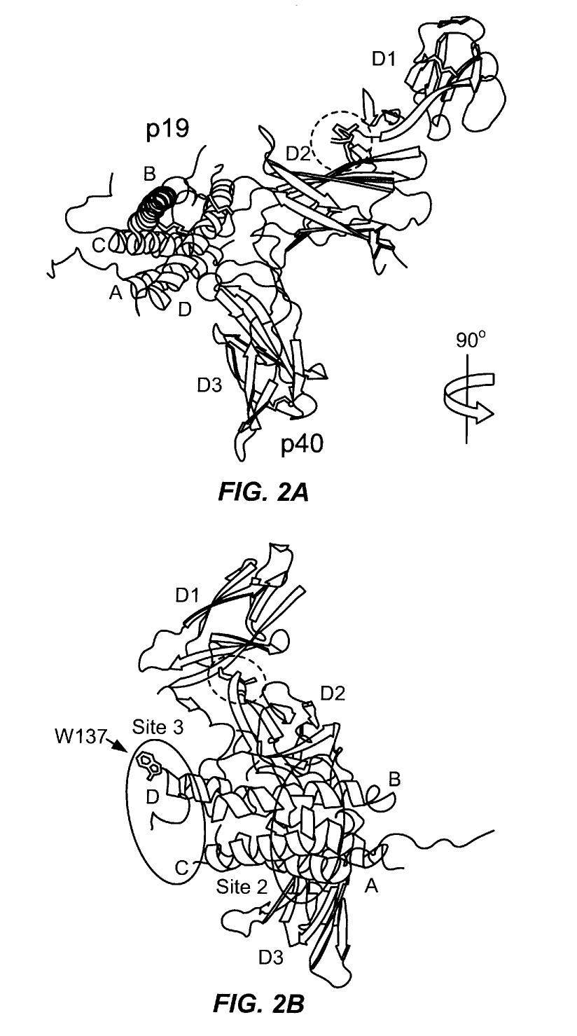

The overall structure of IL-23 is typical of class I cytokine receptor complexes and strongly resembles the p35/p40 heterodimer structure of IL-12 (discussed below) (FIG. 2). The p40 subunit, as previously seen, is composed of three domains (D1, D2 and D3) of approximate dimensions 100×45×25 Å, and superimposes with the previous human p40 structure with an overall root mean square deviation (RMSD) of 1.3 Å. The D1 domain is an S-type Ig-fold in which strand A is swapped from the three-strand sheet so that it now joins the four-strand sheet. The D1 domain interacts with the D2 domain in an unusual fashion in which the long axis of the domain is displaced from co-linearity with the D2 domain, such that it engages the top of D2 in a nearly orthogonal orientation (FIG. 2b), as opposed to the more conventional end-to-end packing of tandem Ig domains, as seen for the D2 and D3 domains. The D2 and D3 domains represent the canonical CHR found in all class I cytokine receptors, with the conserved disulfide bonds in D2 (C 109-120 and C 148-C 171). We observed one GlcNAc moiety emanating from Asn200 on the D2 domain that packs against the D1 domain, but at a location distant from the cytokine binding site (FIG. 2). The remaining consensus Asn-linked glycosylation sites in p40 are unmodified or the glycans disordered in the structure. The D3 domain contains the conserved WSXWS motif (WSEWA in p40) on the G strand. This consensus sequence is a part of a β-bulge and p-cation stacking motif in which side chains from the F strand (R287 and Q289) stack between aromatic residues on the WSEWA motif.

### Example 3

### IL-23 p19 Structure

The four-helix bundle p19 is topologically similar to other canonical long chain four-helix cytokines (FIGS. 2a and 2b), which are so far the only known protein structures to exhibit an up-up-down-down helix topology. As is typical in cytokine structures, the helices are well-ordered, while several inter-helical loops (residues 29-47 (A-B loop), 92-99 (B-C loop), and 123-137 (C-D loop)) lack defined electron density, which likely indicates flexibility of these regions. A comparison of p19 with all four-helix bundle cytokine structures shows the closest similarity with Interleukin-6 (Somers, 1997 #744) (Dali Z-score of 12.7 with RMSD of 2.5 Å for 117 overlapping residues). p19 has ˜15% sequence identity with p35, and superposition of 107 overlapping residues (out of 133) yields an RMSD of 2.3 Å (Dali Z-score of 10.5). Thus, we speculate that even though p19 and p35 both engage p40, they are not simply paralogs of one another, but may have undergone convergent evolution to engage p40 from distinct four-helix bundle precursors. The mature p19 polypeptide sequence is shorter than mature p35 by 27 residues, which is partially manifested in the structure by the truncation of the p19 A, C and D helices by two, one and two helical turns, respectively, compared to p35. A major deviation between the p19 and p35 structures is seen at the C-terminal end of the A-B loop in p35, which forms an 11-amino acid disulfide-bonded loop (missing in p19) that forms two turns of an a-helix and forms numerous interactions with p40.

### Example 4

### IL-23 p19/p40 Interface

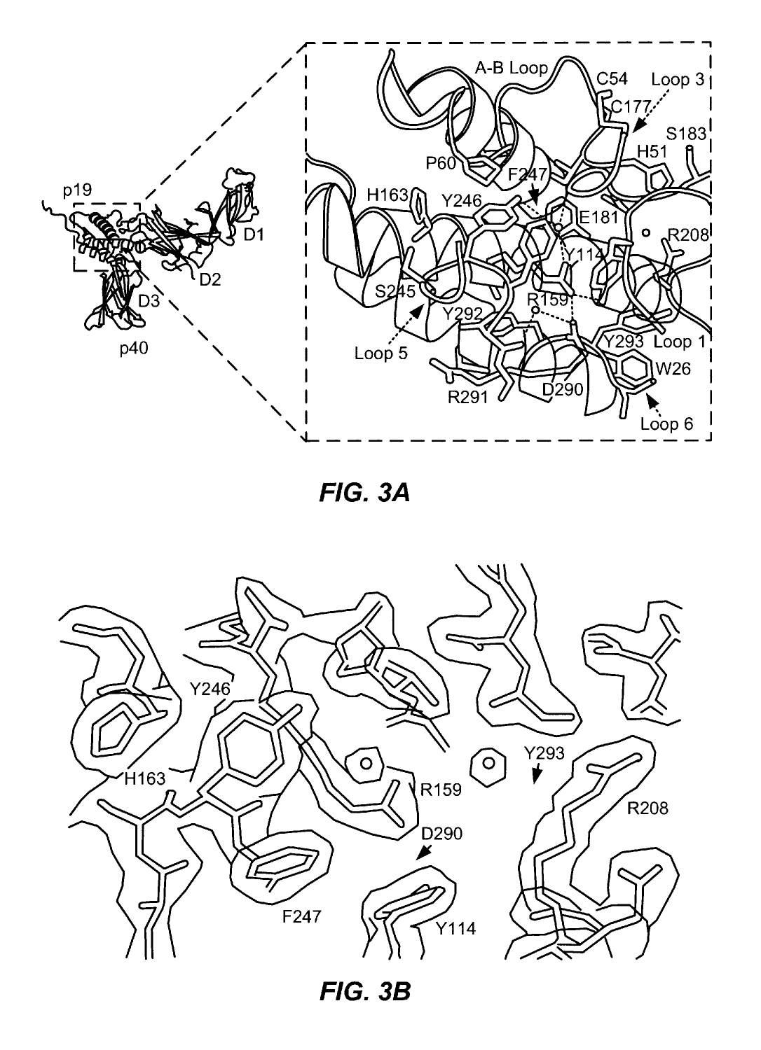

In the IL-23 interface, p40 loops 1 and 3 from the D2 domain, and loops 5 and 6 from D3 interact with the p19 A and D helices, as well as second half of the long AB-loop (FIG. 3a). The inter-subunit disulfide bond unique to the IL-12/23 cytokines is formed at the top edge of the interface between p19 residue Cys54 on the AB-loop to p40 Cys 177. This disulfide bond is peripheral to the main structural epitope between p19 and p40, and in the case of IL-12, was shown not to be necessary for the p35/p40 interaction (Yoon, C., et al. (2000). Embo J 19:3530-41.). There is a large receptor-cytokine interface involving 18 residues of p19 and 14 residues of p40 that buries 1730 Å² of surface area, with 830 Å² buried on p19 and 900 Å² buried on p40.

The p40 binding site forms a ‘volcano-like’ surface with Asp290 at the base of the crater, which is lined with hydrophobic residues such as Tyr246, Phe247, Tyr292, Tyr293 (FIG. 3a). A peripheral group of both apolar and hydrogen bonding residues (for example Arg208, Ser245, Glu181, Arg291) form a ring of ‘peaks’ surrounding the crater. In the heart of the interface, p19 extends Arg159 from its helix D into the crater such that its guanidium group forms an extensive network of interactions including a salt bridge to p40 residue Asp290 and a hydrogen bond to Tyr114. The e-nitrogen of the p19 Arg159 guanidinium group also forms water-mediated hydrogen bonds to Tyr246 and Asp290 (FIG. 3a). These waters also hydrogen bond to the main chains of Ala179 of loop 3 and Ser248 of loop 5, respectively.

There are several other ordered water molecules visible at the periphery of the interface that appear to stabilize the p40 interaction (FIG. 3b). In p40, the aromatic residues lining the crater of the Arginine pocket form multiple van der Waals interactions with backbone and side chain atoms on helices A and D of p19 that surround the centrally protruding Arg159 (FIG. 3a). The principal interactions involve p40 Tyr292 packing against the p19 D-helix main chain and forming hydrophobic contacts with p19 Ala155 and 158, and p40 Tyr293 interacting with p19 D-helix Ala152 and A-helix Trp26. The outermost shell of the interface exhibits several additional side-chain specific hydrogen bonds from p40 residues Glu181 and Ser245, which interact with p19 residues His51 from the p19 AB-loop, and His 163, respectively (FIG. 3a). Collectively, the p19/p40 interface appears to have three shells of interactions: a charged center at the base of the crater, hydrophobic contacts lining the crater, and hydrogen bonded peaks at the interface periphery. Thus, rather than the more conventional interface hotspot where a hydrophobic center is surrounded by polar periphery (Clackson, T. & Wells, J. A. (1995). Science 267:386; Boulager, M. J., et al. (2003). Mol Cell 12:577.), IL-23 has a polar center that appears insulated by a ‘gasket’ of hydrophobic interactions.

### Example 5

### IL-23 Structural Comparison with IL-12

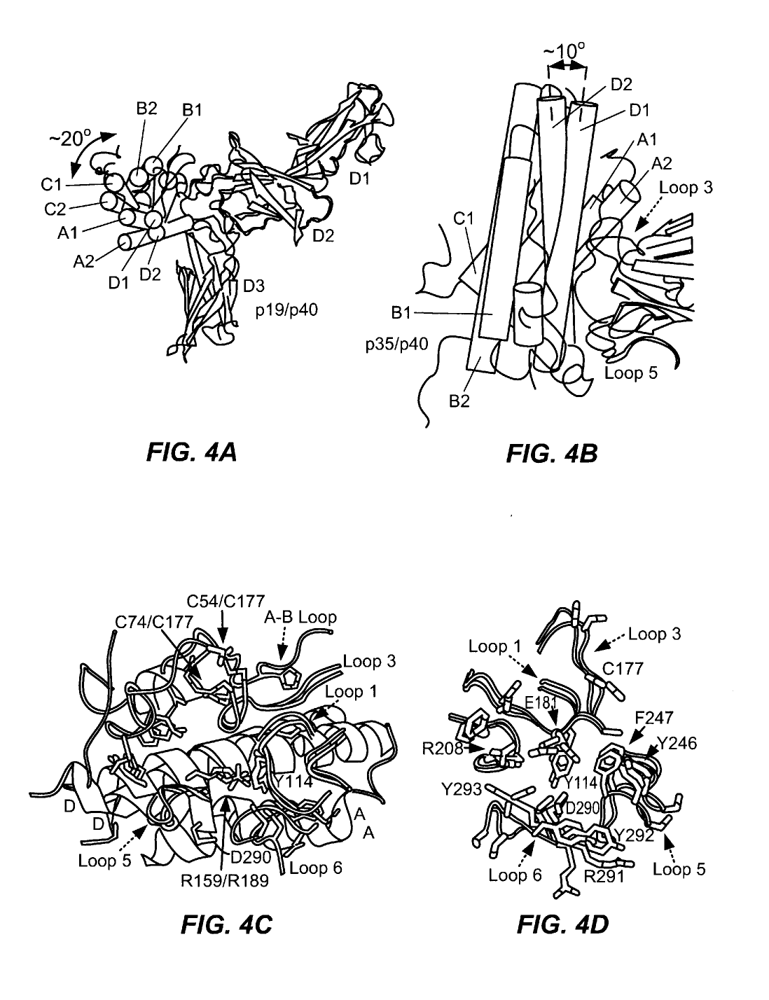

Although both p19 and p35 primarily use their A and D helices to engage p40, there are substantial positional changes with respect to the docking modes on p40. When the p40 molecules from both complexes are superimposed and the complexes are viewed looking down the barrels of the p19 and p3 5 four-helix bundles, it is clear that p19 is rotated clockwise towards the D2 domain of p40 by approximately 20 degrees (FIG. 4a). When viewed from the top, p19 is also tilted towards p40 by approximately 10 degrees compared to p35 (FIG. 4b). The overall result of the rotation and tilt of p19 with respect to p3 5 is that p19 has more intimate interaction between the N -and C-terminal ends of its D and A helices with p40 than p35, and p35 has more intimate interactions with p40 at the C and N-terminal ends of its D and A helices, respectively (FIG. 4b). In this latter region of the IL-12 interface, p40 loops 3 and 5 interdigitate into a ‘corner’ of p35 formed by an II-amino acid disulfide bonded loop C-terminal to Cys74—this mini-loop is missing in p19. Thus, while p35 is less complementary with p40 at one end relative to p19, the rotation and tilt of the four-helix bundle results in improved complementarity at the opposite end.

In comparison to IL-12 (Yoon, C., et al. (2000). Embo J 19:3530.), IL-23 has utilized the same network of charge and hydrogen bonds involving p40 residues Asp290 and Tyr114 at the base of the crater by structurally ‘mimicking’ the Arg189 on the D-helix of p35 with its own p19 D-helix Arg at position 159 (FIG. 4c). While a mutational analysis of interface residues was carried out for IL-12 to assess the energetic importance of p35/p40 contacts in complex assembly (Yoon, C., et al. (2000). Embo J 19:3530.), such data does not currently exist for IL-23. However, instructive comparisons can be made for obviously analogous structural contacts. In IL-12, residues that make up the hydrogen-bonding network involving the central Arg on the cytokine helix D, p40 residues Asp290 and Tyr114, are energetically critical as their mutation abrogates complex formation. Given the vanishing sequence identity between p19 and p35, conservation of this core ‘hotspot’ in both complexes was unexpected. The positional correspondence of these residues (FIG. 4c) and their interactions in the face of highly divergent sequence and docking positions on p40 further suggests convergent structural evolution from distinct precursors to recapitulate the features of this ‘Arginine’ pocket in both heterodimers. The hydrogen bonds in the Arginine pocket in the two complexes are not exactly similar, most likely due to a slight shift in backbone position of the p19 Arg159 versus p35 Arg189, which affects the side chain location and geometry of the resultant bonding network (FIG. 4c). Further direct comparisons of the role of bound water molecules in the respective interfaces is complicated by the differing resolutions of the structures (IL-23 2.3 Å versus IL-12 2.8 Å). Nevertheless, it is clear that the interactions that form the Arginine pocket are structurally conserved, and therefore likely to be important for heterodimer formation in both cytokines.

### Example 6

### Interfacial Interactions of IL-23 and IL-12

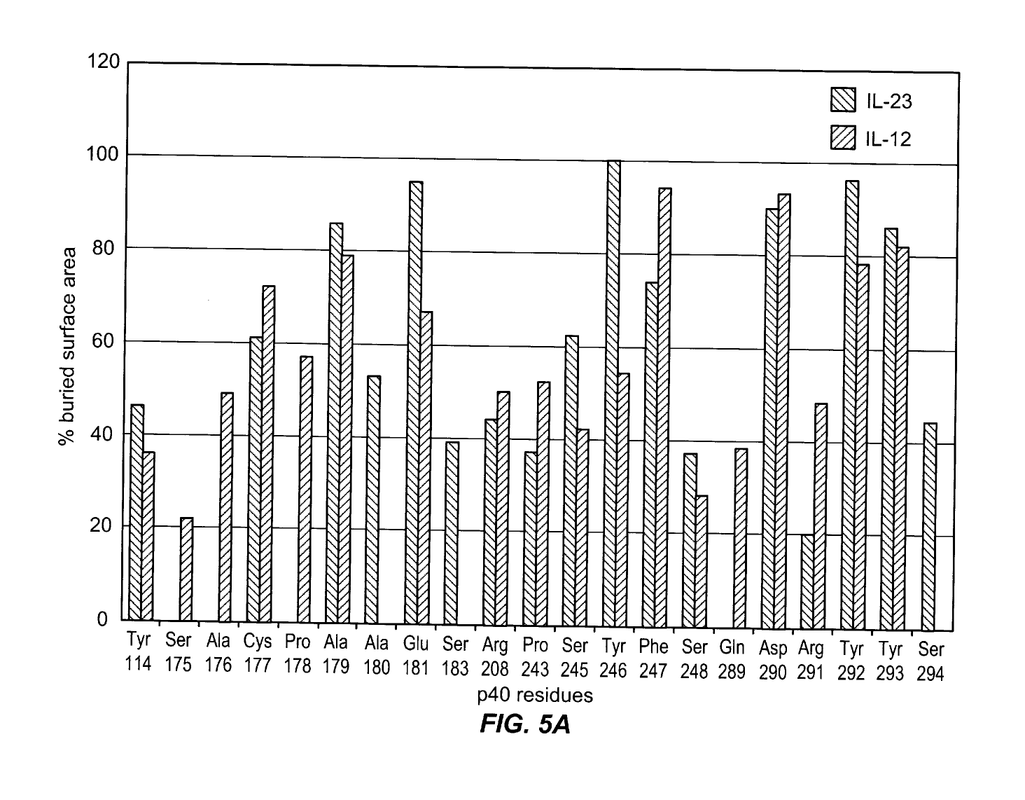

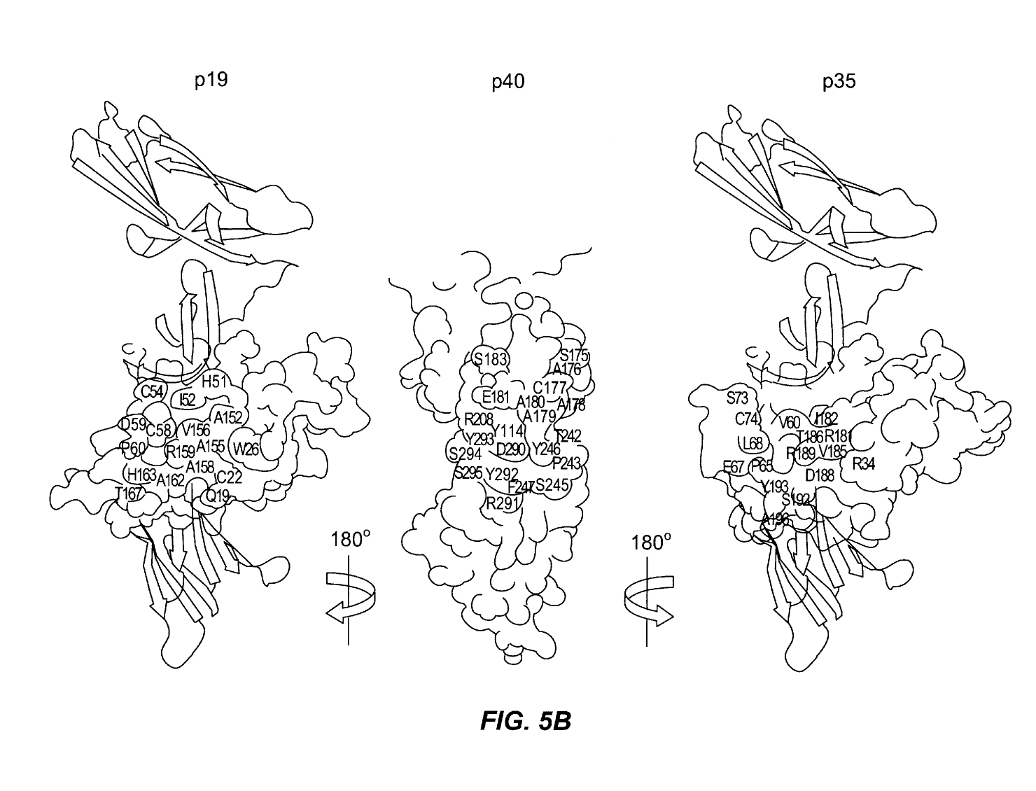

The surrounding interactions in the IL-23 and IL-12 interfaces, however, are almost entirely different (Table 2), which was to be expected given the limited sequence identity between p19 and p35. Comparison of the two interfaces reveals that, with the exception of the central Arginine residue discussed above, there are no other conserved pairwise interactions (Table 2). Many of the same p40 residues are buried within the different interfaces (FIG. 5a), and serve similar structural roles in both complexes, but through interactions with distinct constellations of amino acids on the p19 and p35 surfaces (FIG. 5b). For instance in the interface ‘outer shell’, p40 residue Glu181, which was shown in IL-12 to be energetically-critical for heterodimer formation (Yoon, C., et al. (2000). Embo J 19:3530.), forms a hydrogen bond with Arg183 in p35 versus His51 in p19. Ser245 of p40, whose mutation to Ala had a modest effect in the IL-12 system, hydrogen bonds to the amide nitrogen of Tyr193 in p35 versus the side chain of His163 in p19. In the IL-23 complex, p40 forms several more interactions with the p19 AB-loop near the Cys54-Cys177 interchain disulfide linkage (FIG. 4c), which may be a result of the rotation of p19 towards p40 relative to p35. In IL-12, the AB-loop is largely disordered with the exception of residues C-terminal to the p35 Cys74, and this disorder may be a result of the gap between p35 and p40 above the D helix, formed by the relative rotation of p35 away from p40. Collectively, p40 displays an ability to engage two different cytokine surfaces through a combination of shared (Arg pocket) and distinct interactions (outer shells).

TABLE 2

| p40 | p19 | | H- | p35 | | H- |
|---|---|---|---|---|---|---|
| residue | residue | vdW* | bonds** | residue | vdW* | bonds** |
| Tyr 114 | Arg 159 | 1 | 1 | Arg 189 | 4 | 1 |
| Ser 175 | | 0 | 0 | Ser 73 | 3 | 0 |
| Ala 176 | | 0 | 0 | Ser 73 | 2 | 0 |
| Cys 177 | Cys 54 | 4 | 0 | Ser 73 | 6 | 0 |
| | | | | Cys 74 | | |
| Pro 178 | | 0 | 0 | Cys 74 | 1 | 0 |
| Ala 179 | Asp 59 | 2 | 0 | Val 60 | 3 | 0 |
| | Val 156 | | | Leu 68 | | |
| Ala 180 | Ile 52 | 2 | 0 | | 0 | 0 |
| Glu 181 | His 51 | 18 | 3 | Ile 182 | 11 | 3 |
| | Ile 52 | | | Arg 183 | | |
| | Phe 153 | | | Thr 186 | | |
| Ser 183 | His 51 | 1 | 0 | | 0 | 0 |
| Arg 208 | Ala 152 | 4 | 0 | Ile 182 | 3 | 0 |
| Thr 242 | | 0 | 0 | Leu 68 | 1 | 0 |
| Pro 243 | His 163 | 1 | 0 | Glu 67 | 4 | 0 |
| Ser 245 | Ala 162 | 6 | 1 | Ser 192 | 11 | 1 |
| | his 163 | | | Tyr 193 | | |
| | Thr 167 | | | Ala 196 | | |
| Tyr 246 | Cys 58 | 15 | 1 | Pro 65 | 9 | 1 |
| | Pro 60 | | | Arg 189 | | |
| | Arg 159 | | | | | |
| | His 163 | | | | | |
| Phe 247 | | 0 | 0 | Arg 189 | 2 | 0 |
| Ser 248 | | 0 | 0 | Ser 192 | 1 | 0 |
| Gln 289 | | 0 | 0 | Arg 34 | 2 | 0 |
| Asp 290 | Arg 159 | 1 | 2 | Arg 34 | 2 | 1 |
| Arg 291 | | 0 | 0 | Arg 34 | 2 | 0 |
| Tyr 292 | Gln 19 | 17 | 0 | Arg 34 | 16 | 2 |
| | Cys 22 | | | Arg 181 | | |
| | Ala 155 | | | Asp 188 | | |
| | Ala 158 | | | Arg 189 | | |
| | Arg 159 | | | | | |
| | Ala 162 | | | | | |
| Tyr 293 | Trp 26 | 6 | 0 | Arg 34 | 15 | 1 |
| | Ala 152 | | | Arg 181 | | |
| | | | | Ile 182 | | |
| | | | | Val 185 | | |
| Ser 294 | Trp 26 | 8 | 0 | Arg 34 | 1 | 1 |
| Ser 295 | | 0 | 0 | Arg 34 | 1 | 0 |

### Example 7

### p40 Structural Accommodations for p19 and p35 Binding

The cross-reactivity of p40 is not due to large-scale conformational changes in the binding site. Superposition of p40 from both heterodimers reveals that the backbone conformations of the p40 loops are generally similar, although several of the loops are slightly distorted (FIG. 4d). We interpret this as structural accommodation of the different p19 and p35 cytokine surfaces by somewhat flexible, solvent exposed p40 loops as has been previously reported for receptor-cytokine interactions in the gp130 and IL-4/13 receptor systems (LaPorte, S. L., et al. (2008). Cell 132:259; Boulanger, M. J., et al. (2003). Mol Cell 12:577.), rather than an ‘induced fit’ binding mechanism in which distinct loop arrangements accommodate the divergent surfaces. Strikingly, the center of the Arginine pocket, including residues Asp290, Tyr114, and Phe247 of p40 have preserved their side chain and main chain positions in the two complexes (FIG. 4d). These residues, including Phe247, are energetically essential for formation of the IL-12 complex (Yoon, C., et al. (2000). Embo J 19:3530.). The main conformational deviations seen in the p40 binding site are localized to loop 6 of the p40 D3 domain, where Arg291, Tyr292, and Tyr293 assume different side chain positions. It therefore appears that p40 has preserved the structural context of most of the energetically critical residues used in the IL-12 heterodimer that are also in contact with p19, further supporting their conserved role in IL-23.

### Example 8

### Inter-Chain Contacts and Buried Surface Area of 0.40 Contacting IL-12 and IL-23

To further investigate the basis for the cross-reactivity of p40 for p19 and p35, we analyzed the inter-chain contacts and buried surface area for each p40 residue that makes contact with either p19 or p35 (Table 2 and FIG. 5a). Clearly, p40 uses the same binding surface to contact both p19 and p35 (FIG. 5a). However, when analyzed in terms of inter-atomic contacts (Table 2 and FIG. 5b), of the 23 total p40 residues that make contact with either cytokine, we find that twelve (˜½) of these residues are shared contact residues. Nine of the remaining residues only contact p35, while two only contact p19. The bulk of this disparity reflects the added contact area provided by the A-B loop extension found in p35. The two amino acids that exclusively contact p19 are found at the C-terminal end of loop 3 in p40 and interact with p19 in the ordered section of the A-B loop around the interchain disulfide at Cys54 (discussed earlier). This data is represented graphically in FIG. 5b, with shared residues on the surface representation of p40 (middle panel) colored yellow and p19 and p35-exclusive residues colored pink and green, respectively.

### Example 9

### Structure-Guided Small Molecule/Peptide Antagonist Design for IL-12 and IL-23

As both IL-12 and IL-23 are attractive therapeutic targets for various autoimmune diseases, one goal is to develop small molecule inhibitors of heterodimer formation (Yoon, C., et al. (2000). Embo J 19:3530.). Small molecule antagonism of cytokine-receptor interactions remains a daunting, but important, challenge given how important many cytokine-receptor interactions are in human health and disease. While antibodies can be highly effective antagonists, production costs are high and special storage and handling is required. Despite the immense difficulties in producing small molecule antagonists of protein-protein interactions, one notable exception has been the four-helix cytokine Interleukin-2 (IL-2), for which a panel of high affinity small molecules have been created that antagonize its association with its a-receptor (Thanos, C. D., et al. (2003). Journal of the American Chemical Society 125:15280.). Comparison of the IL-2/drug and IL-2/IL-2Ra complexes revealed that the drug utilizes a similar binding epitope on the cytokine as the receptor (Rickert, M., et al. (2005). Science 308:1477.). Further, an alanine scanning study showed that the drug uses the same energetic hotspots on IL-2 as the receptor, despite inducing conformational changes on the cytokine surface (Thanos, C. D., et al. (2006). PNAS 103:15422.). Given this encouraging proof of concept, small molecule targeting of cytokines remains an active effort in the pharmaceutical industry.

### Example 10

### Structure-Guided Dominant-Negative Antagonist Design for IL-12Rβ1

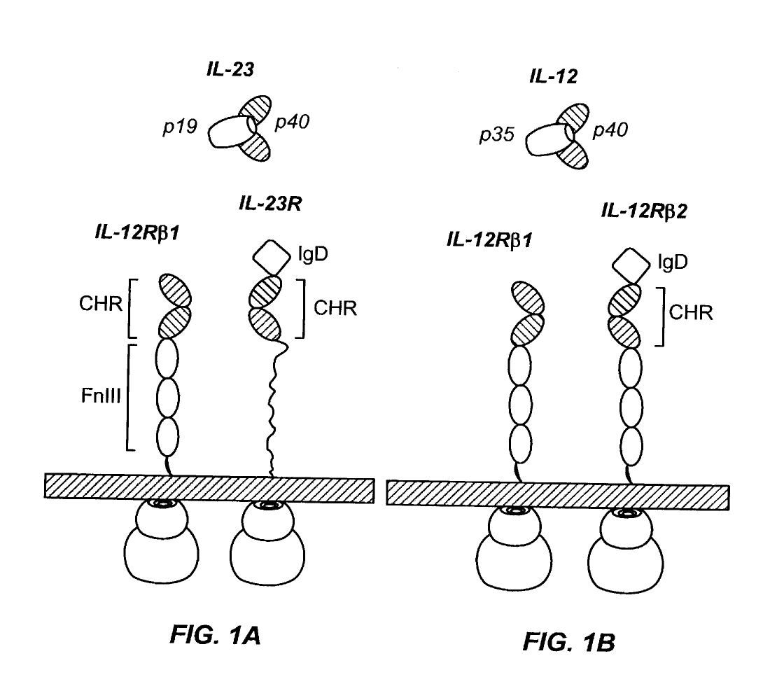

The IL-23 heterodimer engages the signaling receptors IL-12Rβ1 and IL-23R in order to induce a cellular response via the JAK/STAT pathway (FIG. 1). Previous structural studies in the gp130 system provide an architectural template to model the IL-23 quaternary complex (Boulanger, M. J., et al. (2003). Science 300:2101; Boulager, M. J., et al. (2003). Mol Cell 12:577; Chow, D., et al. (2001). Science 291:2150.). Cytokines of the gp130 family possess a ‘site III’ at the tip of the cytokine that engages an Ig-domain on one of the signaling receptors, in addition to the canonical sites I and II on each side of the four-helix bundle. In the p19 structure, the loop connecting the C-D helices contains a Tryptophan (Trp137) residue that is a signature hallmark of the site III interaction (FIG. 2b), as is also seen in members of the gp130-cytokine family (Boulager, M. J., et al. (2003). Mol Cell 12:577; Chow, D., et al. (2001). Science 291:2150.). The mutation or deletion of Trp137 and surrounding residues will generate a cytokine capable of binding IL-12Rβ1 but incapable of binding IL-12Rβ2 or IL-23R. Such a cytokine would function as a dominant negative inhibitor of IL-12 and IL-23 signaling. By analogy with other cytokine-receptor systems that utilize site I, II, and III binding modes, regions in the A-B helices are likely to contribute to IL-23R binding, while regions in the A and C helices are likely to contribute to IL-12Rβ1 binding. Therefore, mutations or deletions in A-B region are useful to generate a cytokine capable of binding IL-12Rβ1 but incapable of binding IL12Rβ2 or IL-23R, while mutations or deletions in the A and C regions are useful for generating a cytokine capable of binding IL-23R but incapable of binding IL-12R β1. These cytokines are able to function as dominant-negative inhibitors of IL-12 and IL-23 signaling.

### Example 11

### Binding of Human IL-23 to Human IL-23R

A) Binding of biotinylated IL-23 to cells transfected with the IL-23 receptor (IL-23R). Baby Hamster Kidney (BHK) cells or other mammalian cells that have been transfected with expression vectors encoding human IL-23R are assessed for their ability to bind biotinylated human IL-23. Cells are harvested with versene, counted and diluted to 10⁷ cells per ml in staining media (SM), which was HBSS plus 1 mg/ml bovine serum albumin (BSA), 10 mM Hepes, and 0.1% sodium azide (w/v). Biotinylated human IL-23 is incubated with the cells on ice for 30 minutes at various concentrations. After 30 minutes, excess cytokine is washed away with SM and the cells are incubated with a 1:100 dilution of streptavidin conjugated to phycoerythrin (SA-PE) for 30 minutes on ice. Excess SA-PE is washed away and cells are analyzed by flow cytometry. The amount of cytokine binding is quantitated from the mean fluorescence intensity of the cytokine staining. Results will demonstrate that human IL-23 bind to human IL-23R-tranfected cells.

B) Inhibition of Specific Binding of Biotinylated Human IL-23 with Unlabeled Cytokine. Binding studies are performed as discussed above, but excess unlabeled human IL-23 is included in the binding reaction. In studies with BHK cells, the amount of unlabeled cytokine is varied over a range of concentrations to find that addition of unlabeled human IL-23 competes for binding of human IL-23 to human IL-23R-transfected cells, indicating that human IL-23 binds to human IL-23R, and supporting the use of this assay for testing antagonistic binding of IL-23 with an IL-23R antagonist.

C) Inhibition of Specific Binding of Biotinylated Human IL-23 with an IL-23R Antagonist. Binding studies are performed as discussed above, except that a range of concentrations of an antagonist of the present disclosure (i.e., a designer cytokine antagonist) to human IL-23R is included in the binding reactions. We expect to find that the anti-human IL-23R antagonist inhibits binding of human IL-23 to human IL-23R-transfected BHK cells, indicating that the anti-human IL-23R antagonist is effective at blocking the binding of IL-23 to the receptor IL-23R.

### Example 12

### Binding of Human IL-12 and IL-23 to Human IL-12Rβ1

A) Binding of biotinylated IL-12 or IL-23 to cells transfected with the IL-12 receptor (IL-12R1). Baby Hamster Kidney (BHK) cells or other mammalian cells that have been transfected with expression vectors encoding human IL-12Rβ1 are assessed for their ability to bind biotinylated human IL-12 or IL-23. Cells are harvested with versene, counted and diluted to 107 cells per ml in staining media (SM), which was HBSS plus 1 mg/ml bovine serum albumin (BSA), 10 mM Hepes, and 0.1% sodium azide (w/v). Biotinylated human IL-12 or IL-23 are incubated with the cells on ice for 30 minutes at various concentrations. After 30 minutes, excess cytokine is washed away with SM and the cells are incubated with a 1:100 dilution of streptavidin conjugated to phycoerythrin (SA-PE) for 30 minutes on ice. Excess SA-PE is washed away and cells are analyzed by flow cytometry. The amount of cytokine binding is quantitated from the mean fluorescence intensity of the cytokine staining. Results will demonstrate that human IL-12 or IL-23 bind to human IL-12Rβ1-tranfected cells.

B) Inhibition of Specific Binding of Biotinylated Human IL-12 or IL-23 with Unlabeled Cytokine. Binding studies are performed as discussed above, but excess unlabeled human IL-12 or IL-23 is included in the binding reaction. In studies with BHK cells, the amount of unlabeled cytokine is varied over a range of concentrations to find that addition of unlabeled human IL-12 or IL-23 competes for binding of human IL-12 or human IL-23 to human IL-12Rβ1-transfected cells, indicating that human IL-12 and human IL-23 bind to human IL-12Rβ1, and supporting the use of this assay for testing antagonistic binding of IL-12 or IL-23 with an IL-12Rβ1 antagonist.

C) Inhibition of Specific Binding of Biotinylated Human IL-12 or IL-23 with an IL-12Rβ1 Antagonist. Binding studies are performed as discussed above, except that a range of concentrations of an antagonist of the present disclosure (that is a designer cytokine antagonist) to human IL-12Rβ1 is included in the binding reactions. We expect to find that the anti-human IL-12Rβ1 antagonist inhibits binding of human IL-12 or IL-23 to human IL-12Rβ1-transfected BHK cells, indicating that the anti-human IL-12Rβ1 antagonist is effective at blocking the binding of IL-12 or IL-23 to the receptor IL-12Rβ1.

### Example 13

### Bioassay for Neutralization of Human IL-23 Mediated IL-17A and IL-17F Production in Murine Splenocytes

Recombinant human IL-23 (rhIL-23) induces the production of IL-17A and IL-17F in murine splenocytes. To evaluate antagonists to IL-23, we examine the neutralization of IL-17A and IL-17F production in rhIL-23 treated murine splenocytes. Antagonists to rhIL-23 are compared to the commercial neutralizing antibody anti-IL-12p40 (Pharmingen, Franklin Lakes, N.J.) or to an antibody against IL-23R (Santa Cruz Biotechnology).

Experimental protocol: a single cell suspension of splenocytes is prepared from whole spleens harvested from either C57BL/6 or BALB/c mice. After red blood cell lysis with ACK buffer (0.010 M KHCO3, 0.0001 M EDTA, 0.150 M NH4Cl), splenocytes are washed and resuspended in RPMI buffer (containing 1% non-essential amino acids, 1% Sodium Pyruvate, 2.5 mM HEPES, 1% L-glutamine, 0.00035% 2-mercaptoethanol, 1% Pen/Strep, 10% FCS and 50 ng/ml human IL-2 (R&D Systems, Minneapolis, Minn.)). Cells are seeded at 500,000 cells per well in a 96-well round bottom plate. In a separate plate, rhIL-23 at a concentration of 10 pM is pre-incubated for 30-90 minutes at 37° C. with 3-fold serial dilutions of the IL-12Rβ1 antagonists or the IL-23R antagonists of the present disclosure (i.e. a designer cytokine antagonist) at various concentrations. The IL-23 ligand plus antagonists are then added to the splenocytes and incubated at 37° C., 5% CO2 for 24-72 hours. The supernatants are collected and frozen at −80° C. until ready to process. The levels of IL-17A and IL-17F protein in the supernatants are measured using bead-based sandwich ELISAs. A commercial kit (Upstate, Charlottesville, Va.) is used to measure IL-17A protein. An ELISA using an antibody to IL-17F (Abcam) conjugated to a bead is used to measure IL-17F. IC50 values for each antagonist are calculated as the amount of antagonist needed to neutralize 50% of the activity of rhIL-23.

Expected Results:

In the presence of rhIL-23, the antagonists described herein will be efficacious at reducing the producing of IL-17A, IL-17F, or a combination thereof.

### Example 14

### Plate-Based Binding Assay for 12Rβ1

Materials and Methods: COSTAR® (#9018) 96-well plates are coated with 50 μl IL-12Rβ1 at approximately 4 ug/ml in 0.1 M NaHCO₃, pH 9.6, overnight at 4° C. The next day, plates are washed three times with 0.1% Tween-20IPBS (PBST). Each well is filled with 350 μl of 2% milk (#170-6404, Bio-Rad)/PBST for one hour at RT for blocking. Assay plates are then washed three times with PBST. In binding neutralization experiments, each well is filled with 50 μl of 2% milk/PBST followed by varying concentrations of IL-12Rβ1 antagonists to incubate for one hour at RT and then plates are washed again three times with PBST. In binding or binding-neutralization experiments, each well is the filled with 50 μl of 2% milk/PBST, followed by the addition of 25 μl of IL-12 or IL-23 supernatant. Wells are mixed and then incubated for one hour at RT. Plates are washed three times with PBST. For IL-12 detection, 50 μl of approximately (1:4000) anti-IL-12 antibody in 2% milk/PBST was added to each well for one hour at RT, washed, and then followed with HR-conjugated secondary antibody in 2% milk/PBST. For IL-23 detection, 50 μl of approximately (1:4000) anti-IL-23 antibody in 2% milk/PBST is added to each well for one hour at RT, washed, and then followed with HRP-conjugated secondary antibody in 2% milk/PBST. Alteratively, when His-tagged IL-12 or IL-23 are used as the binding reagents, anti -His-HRP antibodies are used as the detection reagents as detailed above, but without use of a separate HRP-conjugated secondary antibody. In either case, plates are washed three times with PBST and 50 μl of TMB (TMBW-1000-01, BioFX Laboratories) is added to each well to develop for 20-30 min, followed by the addition of 50 μl of stop buffer (STPR-1000-01, BioFX Laboratories) to quench the reaction. Plates are then read at 450 nm on a plate reader.

### Example 15

### Plate-Based Binding Assay for IL-23R

Materials and Methods: COSTAR® (#9018) 96-well plates are coated with 50 ul IL-23R at approximately 4 ug/ml in 0.1M NaHCO₃, pH 9.6, overnight at 4° C. The next day, plates are washed three times with 0.1% Tween-20/PBS (PBST). Each well is filled with 350 ul of 2% milk (#170-6404, Bio-Rad)/PBST for one hour at RT for blocking. Assay plates are then washed three times with PBST. In binding neutralization experiments, each well is filled with 50 ul of 2% milk/PBST followed by varying concentrations of IL-23R antagonists to incubate for one hour at RT and then plates are washed again three times with PBST. In binding or binding-neutralization experiments, each well is the filled with 50 ul of 2% milk/PBST, followed by the addition of 25 ul of IL-23 supernatant. Wells are mixed and then incubated for one hour at RT. Plates are washed three times with PBST. For IL-23 detection, 50 ul of approximately (1:4000) anti-IL-23 antibody in 2% milk/PBST is added to each well for one hour at RT, washed, and then followed with HRP-conjugated secondary antibody in 2% milk/PBST. Alternatively, when His-tagged IL-23 is used as the binding reagents, anti-His-HRP antibodies are used as the detection reagents as detailed above, but without use of a separate HRP-conjugated secondary antibody. In either case, plates are washed three times with PBST and 50 ul of TMB (TMBW-1000-01, BioFX Laboratories) is added to each well to develop for 20-30 min, followed by the addition of 50 ul of stop buffer (STPR-1000-01, BioFX Laboratories) to quench the reaction. Plates are then read at 450 nm on a plate reader.

### Example 16

### Efficacy of Antagonists that Bind to IL-12Rβ1 or IL-23R in Irritable Bowl Syndrome (“IBS”): CNS-Directed Pathogenesis

These studies involve a model focusing on primary CNS-directed pathogenesis of IBS which employs stress stimuli to induce symptoms characteristic of IBS. The neonatal psychosocial stress model mimics some clinical features associated with IBS patients including visceral hyperalgesia, diarrhea and stress-sensitivity. Daily separation of the litter from their mothers for 180 minutes each day during postnatal days 4-18 will result in an alteration of maternal behaviour and significantly reduce times of the licking/grooming behaviour. The stress on the neonates results in permanent changes in the CNS resulting in altered stress-induced visceral and somatic pain sensitivity. Colonic motor function in response to stress is enhanced in these animals and preliminary data shows evidence of increased intestinal permeability (Mayer et al., Eur. J. Surg. Suppl. (587): 3-9 (2002)). Treatment with the antagonists of the present disclosure and subsequent analysis of colonic motor function, epithelial permeability and response to stress stimuli could determine efficacy in this animal model of IBS. Decreases in the incidence of symptoms following treatment with these inhibitors indicate usefulness in the treatment of IBS.

### Example 17

### Efficacy of Antagonists of IL-12Rβ1 or IL-23R in Irritable Bowl Syndrome (“IBS”): Primary Gut-Directed Inducers of Stress

This is a model focusing on primary gut-directed inducers of stress (i.e., gut inflammation, infection or physical stress). Animal studies have indicated that low-grade inflammation or immune activation may be a basis for altered motility, and/or afferent and epithelial function of the gut (Mayer et al, Eur. J Surg. Suppl. 587: 3-9 (2002)). In this model, daily colon irritation is produced in neonatal animals (days 8-21) in the form of daily intracolonic injection of mustard oil. Mustard oil is a neural stimulant and has been shown to induce visceral hyperalgesia following intracolonic administration. This model mimics key features of the IBS including visceral hypersensitivity and alteration in bowel habits. Animals also present with diarrhea or constipation, a key feature of IBS patients (Mayer et al., (2002) Eur. J. surg. Suppl. (587): 3); (Kimball et al, (2005) Am. J. Physiol. Gastrointest. Liver Pathol. 288:G 1266). An antagonist of the present disclosure could be delivered to determine changes in the development of symptoms associated with this model. Decreases in the incidence or magnitude of visceral hypersensitivity and altered gut motility following therapeutic treatment with our inhibitors indicate usefulness for these molecules in the treatment of IBS.

### Example 18

### IL-23 Baf3/huIL-23R/huIL-12Rβ1 STAT3 Bioassay

A Baf3/huIL-23R/huIL-12Rβ1 stably- or transiently-transfected cell line is generated by methods well known to those skilled in the art. Baf3/huIL-23R/huIL-12Rβ1 cells are washed two times with assay media (RPMI 1640 with L-Glutamine plus 10% fetal bovine serum, 1% Sodium Pyruvate, and 2 uM β-Mercaptoethanol) before being plated out at 30,000 cells/well in 96-well, round-bottom tissue culture plates. Serial dilutions of recombinant human IL-23 (ZGI CHO material or eBioscience's Insect heterodimer material) are made up in assay media and added to the plates containing the cells and incubated together at 37° C. for 15 minutes. Additionally the assay was also used to measure neutralization of IL-23 activity. A half maximal concentration (EC50, effective concentration at 50 percent) of IL-23 is combined with serial dilutions of anti-human IL-12 p40 monoclonal antibody (Pharmingen), or serial dilutions of IL-12Rβ1 antagonists or IL-23R antagonists and incubated together at 37° C. for 30 minutes in assay media prior to addition to cells. Following pre-incubation, treatments are added to the plates containing the cells and incubated together at 37° C. for 15 minutes. Alternatively, serial dilutions of IL-12Rβ1 antagonists or IL-23R antagonists are added to the plates prior to addition of the half-maximal concentration of IL-23.

Following incubation, cells are washed with ice-cold wash buffer and put on ice to stop the reaction according to manufacturer's instructions (BIO-PLEX® Cell Lysis Kit, BIO-RAD Laboratories, Hercules, Calif.). Cells are then spun down at 2000 rpm at 4° C. for 5 minutes prior to dumping the media. 50 μL/well lysis buffer is added to each well; lysates are pipetted up and down five times while on ice, then agitated on a microplate platform shaker for 20 minutes at 300 rpm and 4° C. Plates are centrifuged at 4500 rpm at 4° C. for 20 minutes. Supernatants are collected and transferred to a new micro titer plate for storage at −20° C.

Capture beads (BIO-PLEX® Phospho-STAT3 Assay, BIO-RAD Laboratories) are combined with 50 μL of 1:1 diluted lysates and added to a 96-well filter plate according to manufacture's instructions (BIO-PLEX® Phosphoprotein Detection Kit, BIO-RAD Laboratories). The aluminum foil-covered plate is incubated overnight at room temperature, with shaking at 300 rpm. The plate is transferred to a microtiter vacuum apparatus and washed three times with wash buffer. After addition of 25 μL/well detection antibody, the foil-covered plate is incubated at room temperature for 30 minutes with shaking at 300 rpm. The plate is filtered and washed three times with wash buffer. Streptavidin-PE (50 μL/well) is added, and the foil-covered plate is incubated at room temperature for 15 minutes with shaking at 300 rpm. The plate is filtered and washed two times with bead resuspension buffer. After the final wash, beads are resuspended in 125 μL/well of bead suspension buffer, shaken for 30 seconds, and read on an array reader (BIO-PLEX , BIO-RAD Laboratories) according to the manufacture's instructions. Data are analyzed using analytical software (BIO-PLEX® MANAGER 3.0, BIO-RAD Laboratories). Increases in the level of the phosphorylated STAT3 transcription factor present in the lysates are indicative of an IL-23 receptor-ligand interaction. For the neutralization assay, decreases in the level of the phosphorylated STAT3 transcription factor present in the lysates are indicative of neutralization of the IL-23 receptor-ligand interaction. IC50 (inhibitory concentration at 50 percent) values are calculated using GraphPad Prism 4 software (GraphPad Software, Inc., San Diego, Calif.) and expressed as molar ratios for each reagent in the neutralization assay.

We expect IL-23 to induce STAT3 phosphorylation in a dose-dependent manner with an EC₅₀ concentration of approximately 30 pM for the eBioscience heterodimer. We expect the IL-12Rβ1 antagonists to neutralize IL-23 induced STAT3 phosphorylation in a dose-dependent manner. We expect the IL-23R antagonists to neutralize IL-23 induced STAT3 phosphorylation in a dose-dependent manner.

### Example 19

### IL-12 and IL-23 Bioassay

Leukopheresis PBMC: To obtain a consistent pool of PBMCs, normal human donors are voluntarily apheresed. The leukopheresis PBMC are poured into a sterile 500 ml plastic bottle, diluted to 400 ml with room temperature PBS+1 mM EDTA and transferred to 250 ml conical tubes. The 250 ml tubes are centrifuged at 1500 rpm for 10 minutes to pellet the cells. The cell supernatant is then removed and discarded. The cell pellets are then combined and suspended in 400 ml PBS+1 mM EDTA. The cell suspension (25 ml/tube) is overlaid onto Ficoll (20 ml/tube) in 50 ml conical tubes (total of 16 tubes). The tubes are centrifuged at 2000 rpm for 20 minutes at room temperature. The interface layer (“buffy coat”) containing the white blood cells and residual platelets is collected, pooled and washed repeatedly with PBS+1 mM EDTA until the majority of the platelets had been removed. The white blood cells are then suspended in 100 ml of ice-cold Cryopreservation medium (70% RPMI+20% FCS+10% DMSO) and distributed into sterile cryovials (1 ml cells/vial). The cryovials are placed in a −80° C. freezer for 24 hours before transfer to a liquid-nitrogen freezer. Apheresis cells processed in this manner contain T cells, B cells, NK cells, monocytes and dendritic cells.

Preparation of PHA blasts: T cells must be activated in order to express the IL-12 receptor and/or IL-23 receptor to be able to respond to IL-12 and IL-23. Cryopreserved leukopheresis PBMC are thawed, transferred to a sterile 50 ml conical tube, washed once with 50 ml of warm RPMI+10% heat-inactivated FBS+1 μg/ml DNAse I (Calbiochem), resuspended in 50 ml of fresh RPMI/FBS/DNAse medium and incubated in a 37° C. water bath for at least 1 hour to allow the cells to recover from being thawed. The cells are then centrifuged and the cell-supernatant discarded. The cell pellet is resuspended in RPMI +10% FBS and distributed into sterile 75 cm² tissue culture flasks (1×10⁷ cells/flask in 40 ml/flask). PHA-L (5 mg/ml stock in PBS) was added to the cells at a final concentration of 5 μg/ml. The cells re then cultured at 37° C. in a humidified incubator for a total of 5 days. The cells re “rested” for some experiments by harvesting the cells on the afternoon of day 4, replacing the culture medium with fresh RPMI+10% FBS without PHA-L (40 ml/flask) and returning the cells to their flasks and incubating at 37° C. the cells in a humidified incubator for the remainder of the 5 day culture period.

IL-12 and IL-23 bioassays: Three in vitro assays for detection of human IL-12 and IL-23 bioactivity on normal human T cells have been established: 1) IFN-gamma and MIP-1 alpha production, 2) proliferation ([³H]-incorporation) and 3) STAT3 activation. Human PHA blasts (activated T cells) are harvested on day 5 of culture, suspended in fresh RPMI+10% FBS and plated at the desired cell number per well in 96 well plates.

For the IFN-gamma production assay, the cells are plated at 1×10⁶/well in flat-bottom 96-well plates. The cells are cultured at 37° C. in a final volume of 200 ul/well with either medium alone, human IL-2 alone (10 ng/ml; R & D Systems), human IL-12 alone (graded doses; Invitrogen), human IL-23 alone (graded doses; ZGI lot #A1806; CHO-derived), anti-human CD28 mAb alone (graded doses; clone 28.2, e-Biosciences), or each cytokine in combination with anti-human CD28 mAb. Triplicate wells are set up for each culture condition. For the IFN-gamma production assay, cell supernatants (120 ul/well) are harvested after 24-48 hours of culturing the cells at 37° C. in a humidified incubator. Human IFN-gamma and MIP-1 alpha concentrations in these supernatants (pooled for each triplicate) are measured using a commercial LUMINEX® bead-based ELISA kit (Invitrogen) following the manufacturer's instructions.

Effects of IL-23 on IFN-gamma and MIP-1 alpha production were enhanced by culturing the cells with plate-immobilized anti-human CD3 mAb (5 μg/ml) and soluble anti-human CD28mAb (1 μg/ml) as well as harvesting the supernatants (120 μl/well) after 48 hrs of culture at 37° C. the cells in a humidified incubator. Human IFN-gamma concentrations in these supernatants (pooled for each triplicate) are measured using a commercial LUMINEX® bead-based ELISA kit (Invitrogen) following the manufacturer's instructions.

For the [³H]-incorporation assay the cells are plated at 2×10⁵ cells/well in U-bottom 96-well plates. The cells are cultured at 37° C. for 72 hours. The cells are pulsed with 1 μCi/well of [³H]-Thymidine (Amersham) for the last 8 hours of this culture period. The cells are then harvested onto glass-fiber filters and the CPMs of [³H] incorporated are quantitated using a beta counter (TOPCOUNT NXT™, Packard).

For each of these above endpoint parameters, effective neutralization of activity mediated by IL-23 is expected to be observed in the presence of IL-12Rβ1 antagonists and/or IL-23R antagonists described herein.

STAT3 Bioassay: For the STAT3 Bioassay the cells were plated at 2×10⁵ cells/well in U-bottom 96-well plates. Serial dilutions of human IL-12 (R&D) or recombinant human IL-23 (ZGI CHO-derived material or eBioscience's Insect heterodimer material) were prepared in assay media (RPMI 1640 with L-Glutamine plus 10% fetal bovine serum), added to the plates [symbol] cells and incubated together at 37° C. for 15 minutes. Additionally, the assay was also used to measure neutralization of IL-12 and IL-23 activity using either commercially-available neutralizing reagents (as “controls”) or the IL-12Rβ1 antagonists or IL-23R antagonsits described herein. A half-maximal concentration (EC₅₀, effective concentration at 50 percent) of IL-12 or IL-23 is combined with serial dilutions of anti-human IL-12 p40 monoclonal antibody (Pharmingen), or the IL-12Rβ1 antagonists or IL-23R antagonists described herein, and incubated together at 37° C. for 30 minutes in assay media prior to addition to cells. Following pre-incubation, treatments re added to the plates containing the cells and incubated together at 37° C. for 15 minutes.

Following incubation, cells are washed with ice-cold wash buffer and put on ice to stop the reaction, according to manufacturer's instructions (BIO-PLEX® Cell Lysis Kit, BIO-RAD Laboratories, Hercules, Calif.). Cells are then spun down at 2000 rpm at 4° C. for 5 minutes prior to removing the media. 50 μl/well lysis buffer is added to each well; lysates were pipetted up and down five times while on ice, then agitated on a microplate platform shaker for 20 minutes at 300 rpm and 4° C. Plates are centrifuged at 4500 rpm at 4° C. for 20 minutes. Supernatants are collected and transferred to a new micro titer plate for storage at −20° C.

Capture beads (BIO-PLEX® Phospho-STAT3 Assay, BIO-RAD Laboratories) are combined with 50 μl of 1:1 diluted lysates and added to a 96-well filter plate according to manufacture's instructions (BIO-PLEX® Phosphoprotein Detection Kit, BIO-RAD Laboratories). The aluminum foil-covered plate is incubated overnight at room temperature, with shaking at 300 rpm. The plate is transferred to a microtiter vacuum apparatus and washed three times with wash buffer. After addition of 25 μL/well detection antibody, the foil-covered plate is incubated at room temperature for 30 minutes with shaking at 300 rpm. The plate is filtered and washed three times with wash buffer. Streptavidin-PE (50 μl/well) is added, and the foil-covered plate is incubated at room temperature for 15 minutes with shaking at 300 rpm. The plate is filtered and washed two times with bead resuspension buffer. After the final wash, beads are resuspended in 125 ul/well of bead suspension buffer, shaken for 30 seconds, and read on an array reader (BIO-PLEX®, BIO-RAD Laboratories) according to the manufacture's instructions. Data were analyzed using analytical software (BIO-PLEX® MANAGER 3.0, BIO-RAD Laboratories).

Increases in the level of the phosphorylated STAT3 transcription factor present in the lysates are indicative of an IL-12 or IL-23 receptor-ligand interaction. For the neutralization assay, decreases in the level of the phosphorylated STAT3 transcription factor present in the lysates were indicative of neutralization of the IL-12 or IL-23 receptor-ligand interaction. IC₅₀ (inhibitory concentration at 50 percent) values are calculated using GRAPH PAD PRISM® 4 software (GraphPad Software, Inc., San Diego, Calif.) and expressed as molar ratios for each reagent and/or neutralizing entity in the neutralization assay.

IL-12 and IL-23 are both expected to induce STAT3 phosphorylation in a dose-dependent manner with variation from donor to donor in PHA-activated human T cells. IL-12 and IL-23 are both expected to be neutralized by the anti-human IL-12 p40 monoclonal antibody, or by the IL-12Rβ1 antagonists described herein.

Additional bioassays for IL-12 and IL-23 can be found in (Nature 2008; Nat Immunol 9:641; Nat Immunol 9:650; JEM 8:1849; Nat Immunol 8:942; and references therein).

### Example 20

### Measurement of Binding Affinities of IL-12 Rβ1 Antagonists Via Surface Plasmon Resonance (BIACORE®)

IL-12Rβ1 antagonists described herein are evaluated for their binding affinities to IL-12Rβ1 using surface plasmon resonance and BIACORE T-100® instrument (GE Healthcare). Recombinant human IL-12Rβ1 is immobilized to the sensor chip, followed by passing the antagonists over the immobilized ligand to attain affinity measurements.

To determine the best conditions for immobilization, a series of pH scouting experiments are performed. For these experiments; recombinant human IL-12Rβ1 is diluted in five different immobilization buffers; Acetate—4.0, Acetate—4.5, Acetate—5.0, Acetate—5.5, and Borate—8.5. Using Immobilization Scouting Wizard, these conditions are tested and the best condition for immobilization is determined.

For the immobilization procedure, IL-12Rβ1 protein is diluted in the appropriate buffer as defined above, and then immobilized onto a Series S Sensor Chip (CM5, GE Healthcare/BIACORE® #BR-1006-68) using the amine coupling kit and BIACORE® Immobilization Wizard. Briefly, the level of immobilization is targeted to 300 BIACORE® Resonance Units (RU), and IL-12Rβ1 is only injected over an active flow cell. After the immobilization procedure, active sites on the flow cell are blocked with ethanolamine. Non-specifically bound protein is removed by washing with 50 mM NaOH. The final immobilization level is 466RU. The reference cell is activated and then blocked with ethanolamine.

To determine the optimal contact time, RL (resonance signal of the ligand) testing is performed. Control proteins, soluble IL-23 and IL-12 are diluted in 1× HSB-EP+ running buffer (GE Healthcare/Biacore, #BR-1006-69). The IL-12 or IL-23 are injected over both the active and reference cells for three different contact times. From this RL testing, the contact time for the IL-12 and IL-23 will be determined.

For the kinetic run, serial dilutions of the control proteins, IL-12 or IL-23, or the IL-12Rβ1 antagonists described herein, are prepared in 1× HBS-EP+ buffer. Duplicate injections of each concentration are performed. The analyte injections are at approximately 30 μl/min. [symbol] ions are also performed to allow for subtraction of instrument noise and drift.

Regeneration buffers supplied in the Regeneration Scouting Kit (GE Healthcare/BIACORE® #BR-1005-56) are used to determine regeneration conditions. This run is performed manually, starting from the least mild condition. At the end of this procedure, the optimal regeneration condition is determined.

As a check for IL-12Rβ1 specific interactions, similar procedures can be performed using immobilized recombinant human IL-23R or IL-12Rβ2.

Data are analyzed using BIACORE® Evaluation software (v.1.1.1) to define the kinetic values of the interaction of IL-12Rβ1 with the control proteins IL-23 or IL-12 and the IL-12Rβ1 antagonists described herein. Baseline stability is assessed to ensure that the regeneration step provided a consistent binding surface throughout the sequence of injections. Binding curves are normalized by double-referencing, and duplicate injection curves are checked for reproducibility. The resulting binding curves are globally fit to the bivalent interaction model. On- and off-rate measurements can also be determined by data analysis using methods known to those skilled in the art.

We expect the data to demonstrate moderate to high affinity binding of human IL-12Rβ1 to the control proteins, IL-12 and IL-23 as well as to the IL-12Rβ1 antagonists described herein.

### Example 21

### Measurement of Binding Affinities of IL-23R Antagonists to IL-23R Via Surface Plasmon Resonance (BIACORE®)

IL-23R antagonists described herein are evaluated for their binding affinities to IL-23R using surface plasmon resonance and BIACORE® T-100 instrument (GE Healthcare). Recombinant human IL-23R is immobilized to the sensor chip, followed by passing the antagonists over the immobilized ligand to attain affinity measurements.

To determine the best conditions for immobilization, a series of pH scouting experiments are performed. For these experiments, recombinant human IL-23R is diluted in five different immobilization buffers; Acetate—4.0, Acetate—4.5, Acetate—5.0, Acetate—5.5, and Borate—8.5. Using Immobilization Scouting Wizard, these conditions are tested and the best condition for immobilization is determined.

For the immobilization procedure, IL-23R protein is diluted in the appropriate buffer as defined above, and then immobilized onto a Series S Sensor Chip (CM5, GE Healthcare/BIACORE® #BR-1006-68) using the amine coupling kit and BIACORE® Immobilization Wizard. Briefly, the level of immobilization is targeted to 300 BIACORE® Resonance Units (RU), and IL-23R is only injected over an active flow cell. After the immobilization procedure, active sites on the flow cell are blocked with ethanolamine. Non-specifically bound protein is removed by washing with 50 mM NaOH. The final immobilization level was 466RU. The reference cell is activated and then blocked with ethanolamine.

To determine the optimal contact time, RL (resonance signal of the ligand) testing is performed. Control proteins, soluble IL-23, is diluted in 1× HSB-EP+ running buffer (GE Healthcare/Biacore, #BR-1006-69). The IL-23 is injected over both the active and reference cells for three different contact times. From this RLtesting, the contact time for the IL-23 will be determined.

For the kinetic run, serial dilutions of the control proteins IL-23, or the IL-23R antagonists described herein, are prepared in IX HBS-EP+buffer. Duplicate injections of each concentration are performed. The analyte injections are at approximately 30 ul/min. Buffer injections are also performed to allow for subtraction of instrument noise and drift.

Regeneration buffers supplied in the Regeneration Scouting Kit (GE Healthcare/BIACORE® BR-1005-56) are used to determine regeneration conditions. This run is [symbol] anually, starting from the least mild condition. At the end of this procedure, the optimal regeneration condition is determined.

As a check for IL-23R specific interactions, similar procedures can be performed using immobilized recombinant human IL-12Rb1 or IL-12Rβ2.

Data are analyzed using BIACORE® Evaluation software (v.1.1.1) to define the kinetic values of the interaction of IL-23R with the control protein IL-23 and the IL-23R antagonists described herein. Baseline stability is assessed to ensure that the regeneration step provided a consistent binding surface throughout the sequence of injections. Binding curves are normalized by double-referencing, and duplicate injection curves are checked for reproducibility. The resulting binding curves are globally fit to the bivalent interaction model. On- and off-rate measurements can also be determined by data analysis using methods known to those skilled in the art.

Moderate to high affinity binding of human IL-23R to the control protein IL-23 as well as to the IL-23R antagonists described herein is expected.

### Example 22

### Expression and Purification of IL-23 and Mutein and Deletion Variants from Insect Cells

Recombinant IL-23 or IL-23 muteins or IL-23 deletion polypeptides (including designer cytokine antagonists) are expressed using the baculovirus system. High-titer baculovirus stocks are prepared by transfection and amplification in Spodoptera frugiperda (SF9) cells cultured at 28° C. in SF900 II media (Invitrogen). Protein expression is carried out in Trichopulsia ni (HI-FIVE™, Invitrogen) cells growing in suspension in INSECT XPRESS™ media (Lonza).

Human p19 and p40 cDNAs or muteins or deletion constructs thereof are cloned into the insect cell expression vector pACSG2 (BD Biosciences) in frame with an N-terminal gp67 leader sequence and C-terminal hexa-histidine tag. To generate glycan-minimized IL-23, kifunensine-treated Hi-Five cells are co-infected with p19 and p40 viruses along with virus carrying the endoglycosidase-H (endoH) cDNA, and protein is allowed to express for 48 h. The p19/p40 IL-23 heterocomplex is captured from supernatant using Ni-agarose (Qiagen) with yields approaching 20 mg/L. Ni-purified IL-23 is diluted to 1 mg/mL, subjected to reductive methylation (Walter, 2006 #721), concentrated, and purified by FPLC on a SUPERDEX® 200 16/60 size exclusion column (GE Healthcare) equilibrated in 10 mM Hepes, pH 7.0, 150 mM NaCl. Peak fractions are pooled and concentrated for crystallization.

### Example 23

### Transfection and Expression of IL-23 and IL-23 Mutein and IL-23 Deletion Variant Protein in 293 Cells

IL-23 or IL-23 muteins or IL-23 deletion proteins are produced transiently in 293F cells (Invitrogen, Carlsbad, Calif. Cat #R790-07). Large-scale plasmid DNA is isolated using a PURELINK™ HiPure Plasmid Gigaprep Kit (Invitrogen, Carlsbad, Calif. Cat #K210009) according to manufacturer's instructions. 293F suspension cells are cultured in 293 Freestyle medium (Invitrogen, Carlsbad, Calif. Cat #12338-018) at 37° C., 6% CO₂ in four 3 L spinner flasks at 95 RPM. Fresh medium is added to each spinner immediately prior to transfection to obtain a 1.5 liter working volume at a final density of 1×10⁶ cells/mL. For each spinner, 2.0 mL of LIPOFECTAMINE™ 2000 (Invitrogen, Carlsbad, Calif. Cat #11668-019) is added to 20 mL OPTI-MEM® medium (Invitrogen, Carlsbad, Calif. Cat #31985-070) and 1.5 mg of DNA (constructs containing the appropriate IL-23 p19 and p40 polynucleotides or muteins or deletion polynucleotides thereof) is diluted in a separate tube of 20 mL OPTI-MEM®. For each spinner, the DNA and lipid are incubated separately at room temperature for 5 minutes, then combined and incubated together for an additional 30 minutes at room temperature with occasional gentle mixing. One tube of lipid-DNA mixture is added to each spinner of 293F cells which is returned to 37° C., 6% CO₂ at 75 RPM. After approximately 96 hours, the conditioned medium is harvested, 0.2 μM filtered and submitted for protein purification.

### Example 24

### Purification of Human IL-23 and IL-23 Mutein and IL-23 Deletion Variants for IMAC Capture

Approximately 10 L of 1× media are augmented to 0.02% sodium azide from 1000× stock and put through a UF/DF process wherein the media is first concentrated 10× against 3×0.1 m² 10 kD PELLICON® membranes (Millipore) in a peristaltic pump system. Then the concentrate is diafiltered into phosphate buffered saline via 5 system volume exchanges. Harvested UF/DF media is adjusted to 0.5M NaCl (via addition of 0.4M Solid), 25 mM Imidazole (addition of solid) and the pH adjusted to 7.5 (using HCl). The adjusted concentrate is 0.22 um sterile filtered (Nalgene) and analyzed via RP-HPLC and Western blot for recovery, comparing against 1× media and the permeates. Analyses show that target is nearly completely recovered at this step.

IMAC (Immobilized Metal Affinity Chromatography) Capture-UF/DF media is loaded over 137 mL Ni NTA His Bind Resin (Novagen) packed in a 2 cm glass column (Millipore) at 0.2 mL/min at 4 C. The column is equilibrated in 500 mM NaCl, 50 mM NaPO₄, 25 mM imidaozloe pH 7.5 buffer. Upon complete washout of the applied media, the flow rate is increased to 20 mL/min and the column washed with equilibration buffer until UV at 254 nm and 280 nm is baseline stable. Bound protein is eluted stepwise using a 40 mM and 500 mM imidazole steps, each in equilibration buffer. The elution flow rate is 20 mL/min and 25 mL fractions are collected. Pools are made based on the inflection of A280 nm and analyzed by RP-HPLC, as well as reducing and non-reducing SDS-PAGE coomassie gels. Only the 500 mM pool had target as analyzed by these methods. Protein is completely captured by the IMAC resin, as assessed by Western blot and RP-HPLC.

SUPERDEX® 200 Coarse SEC (Size Exclusion Chromatography)

The 500 mM IMAC pool is expected to be considered pure enough to warrant SEC for final formulation and polishing. The pool is initially concentrated to 50 mL against 1×50 cm² 100 kD MWCO membrane (Millipore) in the Labscale TFF system. At 50 mL, this concentrate is transferred to a 30 kD ULTRACEL® membrane (Millipore). Concentration continues via centrifugation until a final volume of 15 mL is reached. The final concentrate is injected over a 26/60 (318 mL) SUPERDEX® 200 size exclusion column (GE Healthcare) running in 35 mM NaPO₄, 120 mM NaCl pH 7.2 at 3.0 mL/min. 2.5 mL fractions are collected throughout the isocratic elution. A total of three runs are performed, with 6 mL injections per run. Elution fractions are analyzed by SDS-PAGE non-reducing gel, and the resulting pools are analyzed by RP-HPLC. Monomeric protein is selectively pooled.

The pools of protein from the first size exclusion run are slightly contaminated with higher molecular weight species. They are pooled together, and re-concentrated to <10 mL against 63.5 mm YM30 stirred cell membrane (Millipore). The concentrate is reinjected over the SUPERDEX® 200 size exclusion column under identical conditions as before. A conservative pool is made based on the UV absorbance inflection at 280 nm, and assayed by RP-HPLC for a putative target concentration. This final pool is 0.22 μm filtered (Millipore), aliquoted, and stored at −80° C.

### Example 25

### Analysis of Complex Formation Between IL-12Rβ1 and IL-23R Without Extracellular Domain

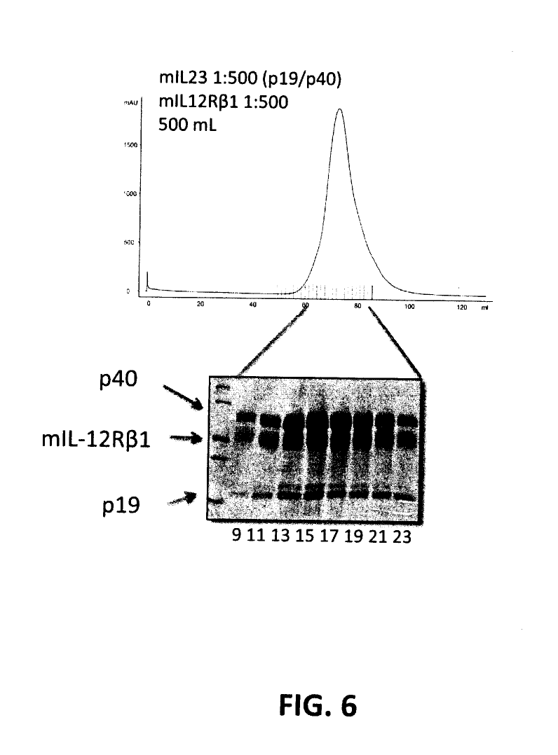

IL-23 (including the p19 and p40 subunit) forms a stable complex with the extracellular domain of IL-12Rβ1 in the absence of the extracellular domain of IL-23R. This stable complex can be observed by gel filtration chromatography as shown in FIG. 6. Ternary complexes with mutant p19 polypeptides that can bind to IL-12Rβ1, but not IL-23R, should therefore form a sink that prevents IL-23 and IL-12 from forming productive signaling complexes that engages IL-12Rβ1 and their respective co-receptor.

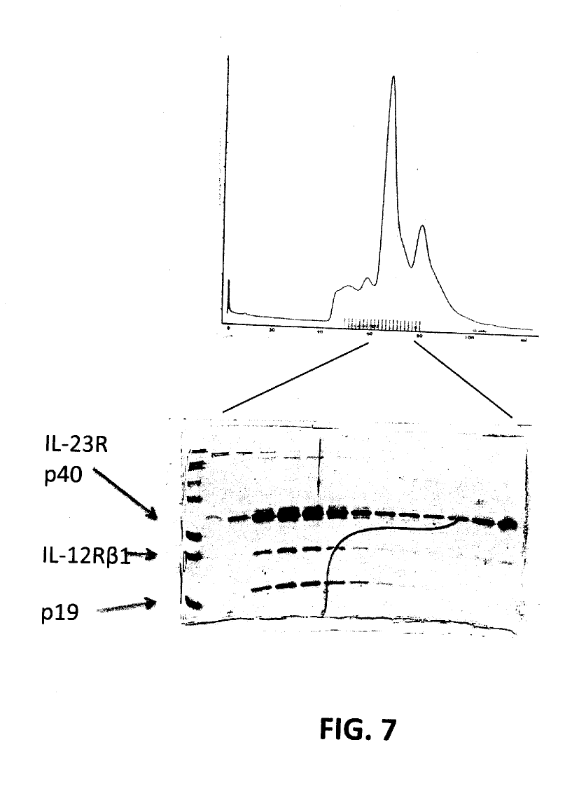

Furthermore, when IL-12Rβ1 and IL-23R extracellular domains are combined with p19 and p40, a stable quaternary complex can be observed by gel filtration as shown in FIG. 7.

Likewise, IL-23 forms a stable complex with the extracellular domain of IL-23R in the absence of the extracellular domain of IL-12Rβ1. This stable complex can be observed by gel filtration chromatography. Ternary complexes with mutant p19 polypeptides that can bind to IL-23R, but not IL-12Rβ1, should therefore form a sink that prevents IL-23 from forming productive signaling complexes that engages IL-23R and IL-12Rβ1.

Equivalents

The invention may be embodied in other specific forms without departing from the spirit or essential characteristics thereof. The foregoing embodiments are therefore to be considered in all respects illustrative rather than limiting on the invention described herein.

What is claimed:

1. An isolated protein comprising a polypeptide sequence at least 90% identical to SEQ ID NO:1, wherein at least one residue selected from the group consisting of residues 130, 131, 132, 133, 134, 135, 136, 137, 138, 139, 140, 141, 142, 143, 144, 145, 146, 147, 148, 149, 150, 151, and 152 is mutated or deleted.

2. The protein of claim 1 wherein the polypeptide sequence is at least 95% identical to SEQ ID NO: 1.

3. The protein of claim 1 wherein at least one residue selected from the group consisting of residues 130, 131, 132, 133, 134, 135, 136, 137, 138, 139, 140, 141, 144, and 145 are mutated.

4. The protein of claim 3 wherein at least two residues selected from the group consisting of residues 130, 131, 132, 133, 134, 135, 136, 137, 138, 139, 140, 141, 144, and 145 are mutated.

5. The protein of claim 3 wherein at least two residues selected from the group consisting of residues 136, 137, 138, 141, and 145 are mutated.

6. The protein of claim 2 wherein residue 137 is mutated to a residue other than alanine.

7. The protein of claim 2 wherein residue 137 is mutated to alanine.

8. The protein of claim 6 wherein residue 137 is mutated to a charged amino acid.

9. The protein of claim 3 wherein one or two residues selected from the group consisting of residues 130, 131, 132, 133, 134, 135, and 136 is deleted.

10. The protein of any preceding claim wherein the at least one amino acid is mutated to a non-conserved residue.

11. The protein of claim 10 wherein the at least one amino acid that is mutated in SEQ ID NO: 1 is a charged amino acid and the amino acid is mutated to an uncharged amino acid or to an amino acid of the opposite charge.

12. The protein of claim 1 wherein no more than five amino acids are mutated from SEQ ID NO: 1.

13. The protein of claim 1 that is identical to SEQ ID NO:1 in the region of residues 1-129 and 153-170.

14. The protein of claim 1 wherein at least one residue selected from the group consisting of residues 29, 30, 31, 32, 33, 34, 35, 36, 37, 38, 39, 40, 41, 42, 43, 44, 45, 46 or 47 is mutated or deleted.

15. The protein of claim 1 wherein at least one residue selected from the group consisting of residues 29, 30, 31, 32, 33, 34, 35, 36, 37, 38, 39, 40, 41, 42, 43, 44, 45, 46 or 47 is mutated to a non-conserved amino acid.

16. The protein of claim 1 wherein at least one residue selected from the group consisting of residues His34, Asp36, or Arg38 is mutated to an uncharged amino acid.

17. An isolated protein comprising a polypeptide sequence at least 90% identical to SEQ ID NO:1, wherein at least one residue selected from the group consisting of residues 29, 30, 31, 32, 33, 34, 35, 36, 37, 38, 39, 40, 41, 42, 43, 44, 45, 46 or 47 is mutated or deleted.

18. The protein of any preceding claim further comprising an IL-23 p40 subunit bound to the isolated polypeptide to form a complex.

19. The protein of claims 1-17 wherein the complex has less than 10% of the activity of a wildtype complex comprising SEQ ID NO: 1 bound to an IL-23 p40 subunit.

20. The protein of claims 1-17 wherein the complex competitively inhibits a wildtype complex comprising SEQ ID NO: 1 bound to an IL-23 p40 subunit from IL-12Rβ1 mediated signaling.

21. The protein of claims 1-17 wherein the complex competitively inhibits IL-23 and/or IL-12 mediated signaling.

22. The protein of claims 1-17 wherein the protein inhibits the production of an inflammatory mediator.

23. The protein of claims 1-17 wherein the complex binds to IL-12Rβ1.

24. The protein of claims 1-17 wherein the complex binds to IL-12Rβ1 with an affinity not more than 50 fold weaker than a wildtype complex comprising SEQ ID NO:1 bound to an IL-23 p40 subunit.

25. The protein of claims 1-17 wherein the complex has a weaker binding affinity for IL-23R than a wildtype complex comprising SEQ ID NO:1 bound to an IL-23 p40 subunit.

26. The protein of claims 1-17 wherein the complex does not detectably bind to IL-23R in the presence of IL-12Rβ1 or binds to IL-23R with at least a 50-fold weaker binding affinity than the wildtype complex in the presence of IL-12Rβ1.

27. The protein of any preceding claim further comprising an Fc domain.

28. The protein of any preceding claim further comprising a signal sequence.

29. A nucleic acid comprising a sequence encoding the protein of any preceding claim.

30. A host cell comprising a vector comprising the nucleic acid of claim 29.

31. A method of providing a protein, the method comprising expressing the nucleic acid of claim 29 in a host cell and recovering the protein.

32. An isolated protein comprising an immunoglobulin heavy chain variable domain and/or an immunoglobulin light chain variable domain, wherein one or both of the variable domains form an antigen binding site that binds to IL-23 p19 at an epitope comprising one or more of amino acids 29-47 or 141-152.

33. The protein of claim 27 or 32 further comprising an Fc domain.

34. The protein of claim 27 or 32 that is homo-bivalent.

35. The protein of claim 32 that comprises human or effectively human framework regions.

36. The protein of claim 27 or 32 that is an IgG molecule.

37. A pharmaceutical composition comprising an effective amount of the composition of any of claims 1-28 and 32-36.

38. A method of inhibiting the production of an inflammatory mediator by a mammalian cell comprising the step of contacting the cell with a polypeptide according to any of claims 1-28 and that binds IL-12Rβ1 and inhibits IL-23R signaling.

39. The method of claim 38, wherein the inflammatory mediator is selected from the group consisting of IL-17A, IL-17F, IL-22, IL-26, CCL20, CCR6, RORC, RORC2, RORγt, IL-1, IL-6, IL-23R, IL-21, IL-2, TNF-α, IL-10, IL-4, IL-13, and a combination thereof.

40. A method of preventing or treating an autoimmune or IL-23 mediated disorder in a subject comprising administering to the subject an effective amount of a composition according to claim 37.

41. The method of claim 40 wherein the administration inhibits the production of an inflammatory mediator in the subject.

42. The method of claim 40 wherein the level of the inflammatory mediator in a bodily fluid is reduced.

43. The method of claim 40 wherein the autoimmune disorder is psoriasis, psoriatic arthritis, psoriatic dermatitis, Crohn's disease, ulcerative colitis, rheumatoid arthritis, uveitis, ankylosing spondylitis, and multiple sclerosis.

44. An isolated protein comprising an isolated polypeptide comprising a sequence at least 90% identical to SEQ ID NO:1, wherein at least one amino acid selected from the group consisting of amino acids 10, 11, 12, 13, 14, 15, 16, 17, 18, 19, 20, 21, 22, 23, 24, 25, 26, or 27 is mutated or deleted.

45. The protein of claim 44 wherein the at least one amino acid is mutated to a non-conserved residue.

46. The protein of claim 44 wherein the amino acid in SEQ ID NO:1 is a charged amino acid and the amino acid is mutated to a hydrophobic amino acid or to an amino acid of the opposite charge.

47. The protein of claim 44 wherein the amino acid in SEQ ID NO:1 is a charged amino acid and the amino acid is mutated to a hydrophobic amino acid or to an amino acid of the opposite charge.

48. The protein of claim 44, wherein, in addition, at least one amino acid selected from the group consisting of amino acids 101, 102, 103, 104, 105, 106, 107, 108, 109, 110, 111, 112, 113, 114, 115, 116, or 117 is mutated or deleted.

49. A protein comprising an isolated polypeptide comprising a sequence at least 90% identical to SEQ ID NO: 1, wherein at least one amino acid selected from the group consisting of amino acids 101, 102, 103, 104, 105, 106, 107, 108, 109, 110, 111, 112, 113, 114, 115, 116, or 117 is mutated or deleted.

50. The protein of any preceding claim wherein no more than five amino acids are mutated from SEQ ID NO: 1.

51. The protein of any preceding claim that is identical to SEQ ID NO: 1 in the region of 1-9, 28-100, and 118-170.

52. The protein of any preceding claim further comprising an IL-23 p40 subunit bound to the isolated polypeptide to form a complex.

53. The protein of claims 44-51 wherein the protein has less than 10% of the activity of a wildtype complex comprising SEQ ID NO:1 bound to an IL-23 p40 subunit.

54. The protein of claims 44-51 wherein the complex competitively inhibits IL-23 mediated signaling and does not substantially inhibit IL-12 mediated signaling.

55. The protein of claims 44-51 wherein the complex competitively inhibits a wildtype complex comprising SEQ ID NO: 1 bound to an IL-23 p40 subunit from IL-23 mediated signaling.

56. The protein of claims 44-51 wherein the protein inhibits the production of an inflammatory mediator.

57. The protein of claims 44-51 wherein the complex binds to IL-23R.

58. The protein of claims 44-51 wherein the complex binds to IL-23R with an affinity not more than 50 fold weaker than a wildtype complex comprising SEQ ID NO:1 bound to an IL-23 p40 subunit.

59. The protein of claims 44-51 wherein the complex has a weaker binding affinity for IL-12Rβ1 than a wildtype complex comprising SEQ ID NO: 1 bound to an IL-23 p40 subunit.

60. The protein of claims 44-51 wherein the complex does not detectably bind to IL-12Rβ1 in the presence of IL-23R or binds to IL-12Rβ1 with at least a 50-fold weaker binding affinity than the wildtype complex in the presence of IL-23R.

61. The protein of claims 44-51 further comprising an Fc domain.

62. The protein of claims 44-51 further comprising a signal sequence.

63. The protein of claim 51 wherein at least one amino acid selected from the group consisting of amino acids 10, 11, 12, 13, 14, 15, 16, 17, 18, 19, 20, 21, 22, 23, 24, 25, 26, or 27 is mutated or deleted.

64. A nucleic acid comprising a sequence encoding the protein of claims 44-51 and 63.

65. A host cell comprising a vector comprising the nucleic acid of claim 64.

66. A method of providing a protein, the method comprising expressing the nucleic acid of claim 64 in a host cell and recovering the protein.

67. An isolated protein comprising an immunoglobulin heavy chain variable domain and/or an immunoglobulin light chain variable domain, wherein one or both of the variable domains form an antigen binding site that binds to IL-23 p19 at an epitope comprising one or more of amino acids 10-27.

68. An isolated protein comprising an immunoglobulin heavy chain variable domain and/or an immunoglobulin light chain variable domain, wherein one or both of the variable domains form an antigen binding site that binds to IL-23 p19 at an epitope comprising one or more of amino acids 101-107 or 108-117.

69. The protein of claims 67 and 68 further comprising an Fc domain.

70. The protein of claims 67 and 68 that is homobivalent.

71. The protein of claims 67 and 68 that comprises human or effectively human framework regions.

72. The protein of claims 67 and 68 that is an IgG molecule.

73. A pharmaceutical composition comprising an effective amount of the composition of any of claims 44-51, 53-63, 67-72.

74. A method of inhibiting the production of an inflammatory mediator by a mammalian cell comprising the step of contacting the cell with a polypeptide according to any of claims 44-51, 53-63, 67-72 such that the production of the inflammatory mediator is inhibited.

75. The method of claim 74, wherein the inflammatory mediator is selected from the group consisting of IL-17A, IL-17F, IL-22, IL-26, CCL20, CCR6, RORC, RORC2, RORγt, IL-1, IL-6, IL-23R, IL-21, IL-2, TNF-α, IL-10, IL-4, IL-13, and a combination thereof.

76. A method of preventing or treating an autoimmune or IL-23 mediated disorder in a subject comprising administering to the subject an effective amount of a composition according to claim 73.

77. The method of claim 73, wherein the administration inhibits the production of an inflammatory mediator in the subject.

78. The method of claim 73, wherein the level of the inflammatory mediator in a bodily fluid is reduced.

79. The method of claim 73, wherein the autoimmune disorder is psoriasis, psoriatic arthritis, psoriatic dermatitis, Crohn's disease, ulcerative colitis, rheumatoid arthritis, uveitis, ankylosing spondylitis, and multiple sclerosis.
